SEQUENCE LISTING (22 sequences)

SEQ ID NO: 1 - DNA, 2074 bp, synthetic construct
  source 1..2074: mol_type = other DNA
caggatccgactacaaggaccacgacggcgactacaaggaccacgacatcgactacaaggacgacgacgacaaggagttgactgtgtacaaagtacagaatcttcagtggacagctgacttctctggggatgtgactttgacctggatgaggcccaaaaaaatgccctctgcttcttgtgtatataatgtctactacagggtggttggagagagcatatggaagactctggagacccacagcaataagacaaacactgtattaaaagtcttgaaaccagataccacgtatcaggttaaagtacaggttcagtgtctcagcaaggcacacaacaccaatgactttgtgaccctgaggaccccagagggattgccagatgcccctcgaaatctccagctgtcactccccagggaagcagaaggtgtgattgtaggccactgggctcctcccatccacacccatggcctcatccgtgagtacattgtagaatacagcaggagtggttccaagatgtgggcctcccagagggctgctagtaactttacagaaatcaagaacttattggtcaacactctatacaccgtcagagtggctgcggtgactagtcgtggaataggaaactggagcgattctaaatccattaccaccataaaaggaaaagtgatcccaccaccagatatccacattgacagctatggtgaaaattatctaagcttcaccctgaccatggagagtgatatcaaggtgaatggctatgtggtgaaccttttctgggcatttgacacccacaagcaagagaggagaactttgaacttccgaggaagcatattgtcacacaaagttggcaatctgacagctcatacatcctatgagatttctgcctgggccaagactgacttgggggatagccctctggcatttgagcatgttatgaccagaggggttcgcccacctgcacctagcctcaaggccaaagccatcaaccagactgcagtggaatgtacctggaccggcccccggaatgtggtttatggtattttctatgccacgtcctttcttgacctctatcgcaacccgaagagcttgactacttcactccacaacaagacggtcattgtcagtaaggatgagcagtatttgtttctggtccgtgtagtggtaccctaccaggggccatcctctgactacgttgtagtgaagatgatcccggacagcaggcttccaccccgtcacctgcatgtggttcatacgggcaaaacctccgtggtcatcaagtgggaatcaccgtatgactctcctgaccaggacttgttgtatgcaattgcagtcaaagatctcataagaaagactgacaggagctacaaagtaaaatcccgtaacagcactgtggaatacacccttaacaagttggagcctggcgggaaataccacatcattgtccaactggggaacatgagcaaagattccagcataaaaattaccacagtttcattatcagcacctgatgccttaaaaatcataacagaaaatgatcatgttcttctgttttggaaaagcctggctttaaaggaaaagcattttaatgaaagcaggggctatgagatacacatgtttgatagtgccatgaatatcacagcttaccttgggaatactactgacaatttctttaaaatttccaacctgaagatgggtcataattacacgttcaccgtccaagcaagatgcctttttggcaaccagatctgtggggagcctgccatcctgctgtacgatgagctggggtctggtgcagatgcatctgcaacgcaggctgccagatctacggatgttgctgctgtggtggtgcccatcttattcctgatactgctgagcctgggggtggggtttgccatcctgtacacgaagcaccggaggctgcagagcagcttcaccgccttcgccaacagccactacagctccaggctggggtccgcaatcttctcctctggggatgacctgggggaagatgatgaagatgcccctatgataactggattttcagatgacgtccccatggtgatagcctgagaattcgt

SEQ ID NO: 2 - DNA, 294 bp, synthetic construct
  source 1..294: mol_type = other DNA
gagttgactgtgtacaaagtacagaatcttcagtggacagctgacttctctggggatgtgactttgacctggatgaggcccaaaaaaatgccctctgcttcttgtgtatataatgtctactacagggtggttggagagagcatatggaagactctggagacccacagcaataagacaaacactgtattaaaagtcttgaaaccagataccacgtatcaggttaaagtacaggttcagtgtctcagcaaggcacacaacaccaatgactttgtgaccctgaggaccccagaggga

SEQ ID NO: 3 - DNA, 291 bp, synthetic construct
  source 1..291: mol_type = other DNA
ttgccagatgcccctcgaaatctccagctgtcactccccagggaagcagaaggtgtgattgtaggccactgggctcctcccatccacacccatggcctcatccgtgagtacattgtagaatacagcaggagtggttccaagatgtgggcctcccagagggctgctagtaactttacagaaatcaagaacttattggtcaacactctatacaccgtcagagtggctgcggtgactagtcgtggaataggaaactggagcgattctaaatccattaccaccataaaaggaaaa

SEQ ID NO: 4 - DNA, 279 bp, synthetic construct
  source 1..279: mol_type = other DNA
gtgatcccaccaccagatatccacattgacagctatggtgaaaattatctaagcttcaccctgaccatggagagtgatatcaaggtgaatggctatgtggtgaaccttttctgggcatttgacacccacaagcaagagaggagaactttgaacttccgaggaagcatattgtcacacaaagttggcaatctgacagctcatacatcctatgagatttctgcctgggccaagactgacttgggggatagccctctggcatttgagcatgttatgaccaga

SEQ ID NO: 5 - DNA, 276 bp, synthetic construct
  source 1..276: mol_type = other DNA
ggggttcgcccacctgcacctagcctcaaggccaaagccatcaaccagactgcagtggaatgtacctggaccggcccccggaatgtggtttatggtattttctatgccacgtcctttcttgacctctatcgcaacccgaagagcttgactacttcactccacaacaagacggtcattgtcagtaaggatgagcagtatttgtttctggtccgtgtagtggtaccctaccaggggccatcctctgactacgttgtagtgaagatgatcccggacagc

SEQ ID NO: 6 - DNA, 279 bp, synthetic construct
  source 1..279: mol_type = other DNA
aggcttccaccccgtcacctgcatgtggttcatacgggcaaaacctccgtggtcatcaagtgggaatcaccgtatgactctcctgaccaggacttgttgtatgcaattgcagtcaaagatctcataagaaagactgacaggagctacaaagtaaaatcccgtaacagcactgtggaatacacccttaacaagttggagcctggcgggaaataccacatcattgtccaactggggaacatgagcaaagattccagcataaaaattaccacagtttcatta

SEQ ID NO: 7 - DNA, 291 bp, synthetic construct
  source 1..291: mol_type = other DNA
tcagcacctgatgccttaaaaatcataacagaaaatgatcatgttcttctgttttggaaaagcctggctttaaaggaaaagcattttaatgaaagcaggggctatgagatacacatgtttgatagtgccatgaatatcacagcttaccttgggaatactactgacaatttctttaaaatttccaacctgaagatgggtcataattacacgttcaccgtccaagcaagatgcctttttggcaaccagatctgtggggagcctgccatcctgctgtacgatgagctggggtct

SEQ ID NO: 8 - DNA, 117 bp, synthetic construct
  source 1..117: mol_type = other DNA
ggtgcagatgcatctgcaacgcaggctgccagatctacggatgttgctgctgtggtggtgcccatcttattcctgatactgctgagcctgggggtggggtttgccatcctgtacacg

SEQ ID NO: 9 - DNA, 165 bp, synthetic construct
  source 1..165: mol_type = other DNA
aagcaccggaggctgcagagcagcttcaccgccttcgccaacagccactacagctccaggctggggtccgcaatcttctcctctggggatgacctgggggaagatgatgaagatgcccctatgataactggattttcagatgacgtccccatggtgatagcctga

SEQ ID NO: 10 - protein, 685 aa, synthetic construct
DYKDHDGDYKDHDIDYKDDDDKELTVYKVQNLQWTADFSGDVTLTWMRPKKMPSASCVYNVYYRVVGESIWKTLETHSNKTNTVLKVLKPDTTYQVKVQVQCLSKAHNTNDFVTLRTPEGLPDAPRNLQLSLPREAEGVIVGHWAPPIHTHGLIREYIVEYSRSGSKMWASQRAASNFTEIKNLLVNTLYTVRVAAVTSRGIGNWSDSKSITTIKGKVIPPPDIHIDSYGENYLSFTLTMESDIKVNGYVVNLFWAFDTHKQERRTLNFRGSILSHKVGNLTAHTSYEISAWAKTDLGDSPLAFEHVMTRGVRPPAPSLKAKAINQTAVECTWTGPRNVVYGIFYATSFLDLYRNPKSLTTSLHNKTVIVSKDEQYLFLVRVVVPYQGPSSDYVVVKMIPDSRLPPRHLHVVHTGKTSVVIKWESPYDSPDQDLLYAIAVKDLIRKTDRSYKVKSRNSTVEYTLNKLEPGGKYHIIVQLGNMSKDSSIKITTVSLSAPDALKIITENDHVLLFWKSLALKEKHFNESRGYEIHMFDSAMNITAYLGNTTDNFFKISNLKMGHNYTFTVQARCLFGNQICGEPAILLYDELGSGADASATQAARSTDVAAVVVPILFLILLSLGVGFAILYTKHRRLQSSFTAFANSHYSSRLGSAIFSSGDDLGEDDEDAPMITGFSDDVPMVIA

SEQ ID NO: 11 - protein, 98 aa, synthetic construct
ELTVYKVQNLQWTADFSGDVTLTWMRPKKMPSASCVYNVYYRVVGESIWKTLETHSNKTNTVLKVLKPDTTYQVKVQVQCLSKAHNTNDFVTLRTPEG

SEQ ID NO: 12 - protein, 97 aa, synthetic construct
LPDAPRNLQLSLPREAEGVIVGHWAPPIHTHGLIREYIVEYSRSGSKMWASQRAASNFTEIKNLLVNTLYTVRVAAVTSRGIGNWSDSKSITTIKGK

SEQ ID NO: 13 - protein, 93 aa, synthetic construct
VIPPPDIHIDSYGENYLSFTLTMESDIKVNGYVVNLFWAFDTHKQERRTLNFRGSILSHKVGNLTAHTSYEISAWAKTDLGDSPLAFEHVMTR

SEQ ID NO: 14 - protein, 92 aa, synthetic construct
GVRPPAPSLKAKAINQTAVECTWTGPRNVVYGIFYATSFLDLYRNPKSLTTSLHNKTVIVSKDEQYLFLVRVVVPYQGPSSDYVVVKMIPDS

SEQ ID NO: 15 - protein, 93 aa, synthetic construct
RLPPRHLHVVHTGKTSVVIKWESPYDSPDQDLLYAIAVKDLIRKTDRSYKVKSRNSTVEYTLNKLEPGGKYHIIVQLGNMSKDSSIKITTVSL

SEQ ID NO: 16 - protein, 97 aa, synthetic construct
SAPDALKIITENDHVLLFWKSLALKEKHFNESRGYEIHMFDSAMNITAYLGNTTDNFFKISNLKMGHNYTFTVQARCLFGNQICGEPAILLYDELGS

SEQ ID NO: 17 - protein, 39 aa, synthetic construct
GADASATQAARSTDVAAVVVPILFLILLSLGVGFAILYT

SEQ ID NO: 18 - protein, 54 aa, synthetic construct
KHRRLQSSFTAFANSHYSSRLGSAIFSSGDDLGEDDEDAPMITGFSDDVPMVIA

SEQ ID NO: 19 - DNA, 98 bp, synthetic construct
  source 1..98: mol_type = other DNA
caggatccgactacaaggaccacgacggcgactacaaggaccacgacatcgactacaaggacgacgacgacaaggagttgactgtgtacaaagtacag

SEQ ID NO: 20 - DNA, 25 bp, synthetic construct
  source 1..25: mol_type = other DNA
gtgaattctcaggctatcaccatgg

SEQ ID NO: 21 - protein, 22 aa, synthetic construct
DYKDHDGDYKDHDIDYKDDDDK

SEQ ID NO: 22 - protein, 663 aa, synthetic construct
ELTVYKVQNLQWTADFSGDVTLTWMRPKKMPSASCVYNVYYRVVGESIWKTLETHSNKTNTVLKVLKPDTTYQVKVQVQCLSKAHNTNDFVTLRTPEGLPDAPRNLQLSLPREAEGVIVGHWAPPIHTHGLIREYIVEYSRSGSKMWASQRAASNFTEIKNLLVNTLYTVRVAAVTSRGIGNWSDSKSITTIKGKVIPPPDIHIDSYGENYLSFTLTMESDIKVNGYVVNLFWAFDTHKQERRTLNFRGSILSHKVGNLTAHTSYEISAWAKTDLGDSPLAFEHVMTRGVRPPAPSLKAKAINQTAVECTWTGPRNVVYGIFYATSFLDLYRNPKSLTTSLHNKTVIVSKDEQYLFLVRVVVPYQGPSSDYVVVKMIPDSRLPPRHLHVVHTGKTSVVIKWESPYDSPDQDLLYAIAVKDLIRKTDRSYKVKSRNSTVEYTLNKLEPGGKYHIIVQLGNMSKDSSIKITTVSLSAPDALKIITENDHVLLFWKSLALKEKHFNESRGYEIHMFDSAMNITAYLGNTTDNFFKISNLKMGHNYTFTVQARCLFGNQICGEPAILLYDELGSGADASATQAARSTDVAAVVVPILFLILLSLGVGFAILYTKHRRLQSSFTAFANSHYSSRLGSAIFSSGDDLGEDDEDAPMITGFSDDVPMVIA

(19) United States
(12) Patent Application Publication
(10) Pub. No.: US 2024/0301030 A1
(43) Pub. Date: Sep. 12, 2024

(54) SORLA MINI-RECEPTOR FOR TREATMENT OF ALZHEIMER'S DISEASE

(71) Applicant: Aarhus Universitet, Aarhus C (DK)

(72) Inventors: Olav Michael Andersen, Aarhus C (DK); Anne Mette Gissel Jensen, Aarhus C (DK)

(21) Appl. No.: 18/570,210

(22) PCT Filed: Jul. 1, 2022
(86) PCT No.: PCT/EP2022/068235

§ 371 Date: Dec. 14, 2023

(30) Foreign Application Priority Data
Jul. 2, 2021 (EP) 21183382.7

(51) Int. Cl. C07K 14/705; A61K 48/00; C12N 15/86

(52) U.S. Cl. CPC C07K 14/70571; A61K 48/005; C12N 15/86; C12N 2750/14143

(57) ABSTRACT

The present invention concerns a polynucleotide construct encoding a mini-receptor of SORLA. The present invention further concerns a vector, a cell and/or a composition comprising said polynucleotide construct. The present invention further concerns a polypeptide representing a mini-receptor of SORLA. The present invention further concerns said vector, said cell and/or said composition comprising said poly nucleotide construct, for use in medicine. The present invention further concerns said vector, said cell and/or said composition comprising said polynucleotide construct, for use in treatment, prevention and/or alleviation of Alzheimer's Disease or a disease or disorder associated with Alzheimer's Disease. The present invention further concerns a method of treating Alzheimer's Disease or a disease or disorder associated with Alzheimer's Disease. The present invention further concerns a method of increasing sAPPα in a cell. The present invention further concerns a method of decreasing Aβ38, Aβ40, and/or Aβ42 in a cell.

### TECHNICAL FIELD

The present invention relates to a vector comprising a polynucleotide, which encodes the FnIII cassette domains, the transmembrane domain and the cytoplasmic tail of the protein SORLA (Sortilin-related Receptor with A Type Repeats). The vector can be used for gene therapy in the treatment, prevention and/or alleviation of Alzheimer's Disease (AD).

### BACKGROUND

The protein SORLA (Sortilin-related Receptor with A Type Repeats) is well known to be associated with Alzheimer's disease (AD). The protein is important for Amyloid Precursor Protein (APP) transport out of the endosomes where amyloidogenic processing of APP into pathogenic fragments (i.e. the Amyloid β-peptide (Aβ)) occurs. This SORLA-assisted transport of APP ensures a decreased cleavage of APP by the β-secretase, thereby reducing the production of the amyloid beta (Aβ) peptides. In AD, Aβ accumulates and forms damaging amyloid plaques within the brain. The ability of SORLA to engage in endosomal recycling is linked to a motif in its cytoplasmic tail (i.e. the FANSHY motif) that is important for interaction with the retromer complex (Fjorback et al. 2012) and which assists to traffics cargo out of endosomes.

The SORL1 gene that encodes SORLA is strongly associated with AD, and loss-of-function gene variants are considered causal for the development of AD (Scheltens et al. 2021). Further, SORLA is important for proper endosomal recycling. AD patients generally exhibit endosomal defects independently of the SORL1 gene status. However, due to the large size of the SORL1 gene, therapy approaches using SORLA are not feasible.

Instead, current pharmacological strategies aim to increase retromer function, in part by using small molecules that stabilize the retromer complex (Mecozzi et al. 2014). Increased retromer stability and thus activity has been demonstrated to decrease the amyloidogenic cleavage of APP, with a concomitant increase in the generation of the non-amyloidogenic product soluble APPα (sAPPα) (Mecozzi et al. 2014).

However, no feasible treatment for AD is available to date and AD remains to be an immense burden to patients and the health care system.

### SUMMARY

Due to its involvement in endosomal processes, increased expression of SORLA would be beneficial for AD patients, as well as for individuals at risk of developing AD. However, due to the large size of the SORL1 gene, any increased expression of the protein cannot be introduced by feasible gene therapy approaches available today, for example using an adeno-associated virus (AAV) vector with limited packaging capacity.

The inventors of the present invention have surprisingly discovered that a truncated version of SORLA can be used in gene therapy approaches to treat AD. Full-length SORLA is unsuitable for gene therapy approaches since the gene encoding full-length SORLA (SORL1) is too large to be inserted into suitable vectors for gene therapy. The inventors of the present invention have achieved to generate a SORLA mini-gene encoding a SORLA mini-receptor compatible with viral gene therapy. The length of the genetic sequence required for expression of said SORLA mini-receptor is small enough to be inserted into vectors suitable for gene therapy and may be referred to as a SORLA mini-gene. Most importantly, the SORLA mini-receptor at the same time retains functions beneficial for the treatment of AD, such as participation in endosomal recycling and the ability to decrease the amyloidogenic processing of APP.

The present invention discloses a mini-receptor of SORLA that comprises a cytoplasmic tail, a transmembrane domain and a part of the luminal region, namely the FNIII cassette domain of SORLA.

The inventors of the present invention have realized that the developed mini-receptor mimics the profile of an endosomal enhancer. In other words, SORLA may be regarded as an endosomal enhancer, and the inventors have realized that the herein disclosed mini-receptor may likewise be regarded as an endosomal enhancer. Most importantly, several advantages are associated with the mini-receptor compared to the full length receptor. Firstly, the mini-receptor does not decrease sAPPα, which is a beneficial property since sAPPα is a non-amyloidogenic product (see Example 2). Secondly, the nucleotide sequence for the mini-receptor (i.e. the mini-gene) is small enough to be packaged into a feasible vector for gene therapy, for example an AAV vector. Thirdly, it is speculated that the effect of retromer-enhancing compounds is through SORLA activity, and using a SORLA mini-receptor for gene therapy might therefore be more specific than broader application of retromer enhancing compounds. It might also be feasible to combine applications of both retromer and SORL1 gene therapies such as the herein disclosed SORLA mini-receptor, possibly in combination with other therapies such as small molecule therapies.

In one aspect, the present invention relates to a polynucleotide construct encoding upon expression a polypeptide P, wherein the polynucleotide comprises or consists of
a. a first polynucleotide encoding upon expression a first polypeptide Q1 comprising or consisting of one or more amino acid sequences selected from the group consisting of SEQ ID NO: 11, SEQ ID NO: 12, SEQ ID NO: 13, SEQ ID NO: 14, SEQ ID NO: 15 and SEQ ID NO: 16, or a biologically active sequence variant thereof wherein the variant is at least 80%, such as at least 90%, such as at least 95%, such as at least 99% identical to said amino acid sequence of SEQ ID NO: 11, SEQ ID NO: 12, SEQ ID NO: 13, SEQ ID NO: 14, SEQ ID NO: 15 or SEQ ID NO: 16; and
b. a second polynucleotide encoding upon expression a second polypeptide Q2 comprising or consisting of the amino acid sequence of SEQ ID NO: 17, or a sequence which is at least 80% identical to SEQ ID: 17, such as at least 90%, such as at least 95%, such as at least 99%; and
c. a third polynucleotide encoding upon expression a third polypeptide Q3 comprising or consisting of the amino acid sequence of SEQ ID NO: 18, or a sequence which is at least 70% identical to SEQ ID: 18, such as at least 75%, such as at least 80%, such as at least 90%, such as at least 95%, such as at least 99%;
wherein the polypeptide P comprises no more than 700 amino acids.

In another aspect, the present invention relates to a polypeptide P, comprising or consisting of
a. a first polypeptide Q1 comprising or consisting of one or more amino acid sequences selected from the group consisting of SEQ ID NO: 11, SEQ ID NO: 12, SEQ ID NO: 13, SEQ ID NO: 14, SEQ ID NO: 15 and SEQ ID NO: 16, or a biologically active sequence variant thereof wherein the variant is at least 80%, such as at least 90%, such as at least 95%, such as at least 99% identical to said amino acid sequence of SEQ ID NO: 11, SEQ ID NO: 12, SEQ ID NO: 13, SEQ ID NO: 14, SEQ ID NO: 15 or SEQ ID NO: 16; and
b. a second polypeptide Q2 comprising or consisting of the amino acid sequence of SEQ ID NO: 17, or a sequence which is at least 80% identical to SEQ ID: 17, such as at least 90%, such as at least 95%, such as at least 99%; and
c. a third polypeptide Q3 comprising or consisting of the amino acid sequence of SEQ ID NO: 18, or a sequence which is at least 70% identical to SEQ ID: 18, such as at least 75%, such as at least 80%, such as at least 90%, such as at least 95%, such as at least 99%;
   wherein the polypeptide P comprises no more than 700 amino acids.

In another aspect, the present invention relates to a vector comprising said polynucleotide construct and/or encoding said polypeptide P.

In a further aspect, the present invention relates to a cell comprising said construct and/or said polypeptide P and/or vector.

In a further aspect, the present invention relates to a composition comprising said construct, polypeptide P, vector and/or cell.

In a further aspect, the present invention relates to said construct, polypeptide P, vector, cell and/or composition for use in medicine.

In a further aspect, the present invention relates to said construct, polypeptide P, vector, cell and/or composition for use in treatment, prevention and/or alleviation of Alzheimer's Disease or a disease or disorder associated with Alzheimer's Disease.

In a further aspect, the present invention relates to a method of treating Alzheimer's Disease or a disease or disorder associated with Alzheimer's Disease, the method comprising administering to an individual in need thereof said construct, polypeptide P, vector, cell and/or composition.

In a further aspect, the present invention relates to the use of said construct, polypeptide P, vector, cell and/or composition for the manufacture of a medicament for the treatment, prevention and/or alleviation of Alzheimer's Disease, or a disease or disorder associated with Alzheimer's Disease.

In a further aspect, the present invention relates to the a method of increasing sAPPα in a cell, the method comprising introducing into a cell the polynucleotide construct of the invention, the vector of the invention, the polypeptide P of the invention and/or the composition of the invention, thus increasing sAPPα in said cell.

In a further aspect, the present invention relates to the a method of decreasing Aβ38, Aβ40, and/or Aβ42 in a cell, the method comprising introducing into a cell the polynucleotide construct of the invention, the vector of the invention, the polypeptide P of the invention and/or the composition of the invention,

thus decreasing Aβ38, Aβ40, and/or Aβ42 in said cell.

### DESCRIPTION OF DRAWINGS

FIG. 1: SORLA mini-receptor

Schematic representation of full-length (SORLA-wt) expressed from SORL1-wt and the SorLA minireceptor (“3Fn mini-receptor” or SORLA-3Fn) expressed from SORL1-3Fn (“mini-gene”) showing domain architectures of both constructs (wt=wild-type). The minireceptor comprises or consists of a polypeptide Q1 comprising one or more than one or all FnIII domains of SORLA, a polypeptide Q2 comprising or consisting of a transmembrane domain of SORLA, and a polypeptide Q3 comprising or consisting of a cytoplasmic tail domain of SORLA, which together may be referred to as polypeptide P. Optionally, the mini-receptor may comprise a tag, for example a FLAG-tag.

FIG. 2: Vector map

Schematic representation of the vector construct.

FIG. 3: Predicted secondary-structure-based alignment of the six 3Fn domains in SORLA

(A) Secondary structure of a 3Fn domain; solution structure.

(B) Schematic structure of folding topology with conserved amino acids.

(C-E) Predicted secondary-structure-based alignment of the 6 3Fn domains of SORLA. Arrows (a-g) represent predicted β-strands and bold amino acids are conserved across the 3Fn domains. Only few amino acids are strictly conserved among different 3Fn domains indicating strong sequence variability. The alignment in panel (C) is continued in panel (D) and further in panel (E), together showing an alignment of the six 3Fn domains of SORLA.

The letters below conserved positions (letters in bold) correspond to p/P (=Pro), W (=Trp), Y (=Tyr), N(=Asn), T (=Thr), L (=Leu), and g (=Gly) at positions with a given amino acid conserved in 5-6 of the domains (capital letters used) or less often conserved (lower case letters used). The symbol ϕ is used to represent an amino acid with a hydrophobic side chain at the given position of the domain sequence (=L/Leu; V/Val; A/Ala; I/IIe; M/Met; F/Phe).

FIG. 4: Western Blot analysis of media and lysate harvested from N2a cells

Representative Western Blot (WB) analysis of cell lysates (A) and conditioned medium (B) from N2a cells transfected with APP in the absence (APP+pcDNA) or in the presence of SORLA-wt (APP+SORLA-wt, transcribed from APP+SORL1-wt) or SORLA-3Fn (APP+SORLA-3Fn, transcribed from APP+SORL1-3Fn). Blots from SDS-PAGE separation of proteins from cell lysates were probed with an antibody against for SORLA (targeting the respective protein product of SORL1-WT including the SORL1-3Fn region; as such targeting both full-length SORLA and the mini-receptor), APP (recognized by an anti-myc antibody against the myc-tag on APP), and β-actin, whereas WBs for conditioned medium samples were probed with an antibody against sAPPα. Each condition was tested in duplicates in at least five independent experiments.

Note the substantial difference in how SORLA-wt and the mini-receptor effect the levels of sAPPα (B; *). The mini-receptor is superior to SORLA-wt in leading to cleaved APP, thus leading to increased sAPPα, and in not decreasing sAPPα. This is beneficial for the AD patient as sAPPα has been reported to be a trophic factor and as such to be important for neuronal survival.

FIG. 5: Densiometric scannings of WB membranes.

Quantification of sAPPα production in cells transfected with SORL1-WT (leading to SORLA-wt expression), relating to the Western Blot in FIG. 4, showed significant decreased sAPPα levels compared to cells without exogenous SORLA-wt (=Blank), whereas SORLA-3Fn (via transfection with SORL1-3Fn) had no effect on sAPPα production (N=4; n=8).

FIG. 6: Quantification of sAPPα and sAPPβ levels using MSD immunoassays.

Using mesoscale discovery (MSD) immunoassay, sAPPα (A) and sAPPβ (B) were quantified, further validating that sAPPα is reduced by the presence of wild-type SORLA (via transfection with SORL1-WT) but is not affected or instead rather increased by the presence of the mini-receptor SORLA-3Fn (via transfection with SORL1-3Fn) (A), compared to the sAPPβ level which was both reduced in the presence of wild-type SORLA (via transfection with SORL1-WT) and in the presence of the mini-receptor (via transfection with SORL1-3Fn).

FIG. 7: Quantification of Aβ40 levels using Aβ40 ELISA assay.

Aβ40 ELISA assay on conditioned medium from five samples from three independent experiments. Both SORLA-wt (via transfection with SORL1-wt) and the mini-receptor SORLA-3Fn (via transfection with SORL1-3Fn) significantly decreased the level of Aβ40 compared to cells not transfected with any exogenous SORLA protein.

FIG. 8: Quantification of Aβ38, Aβ40 and Aβ42 levels using MSD immunoassays.

Aβ38 (A), Aβ40 (B), and Aβ42 (C) quantification of conditioned medium using Meso Scale Discovery (MSD) immunoassay. Aβ production of all three Aβ forms were significantly decreased in the presence of both SORLA-wt (via transfection with SORL1-wt) and the mini-receptor SORLA-3Fn (via transfection with SORL1-3Fn) compared to samples without exogenous SORLA (Blank).

FIG. 9: Stabilization of the retromer complex by the mini-receptor

A) Schematic representation of the core retromer units forming an arch that dimerizes by the VPS29 subunit at the top, and where two VPS26 subunits are also in close contact at the bottom.

B) Dimerizing SORLA mini-receptor stabilizes the retromer complex.

FIG. 10: SORLA mini-receptor biology depends on its Retromer binding site A mutant variant of the mini-receptor with substitution of the FANSHY motif by Alanine residues was generated (FLAG-mini-FANSHY), thus lacking the Retromer binding site. Mutated variant (FLAG-mini-FANSHY) and non-mutated variant (FLAG-mini-WT=SORLA-3Fn) of the mini-receptor were used to transfect cells to demonstrate how said mutation impairs intracellular localization. Significantly more FLAG-mini-FANSHY is found at the cell surface and secreted to the cell medium than FLAG-mini-WT. As the mini-receptors (mutated vs. non-mutated variants) showed a different cellular localization, this suggests that Retromer also plays a role in the endosome trafficking of the mini-receptor.

A) Western blot analysis of lysates from cells transfected with FLAG-mini-WT or FLAG-mini-FANSHY indicated similar expression levels of both variants (representative blots and bar graphs from quantifications of five independent experiments).

B) In contrast, Western blot analysis of lysates from cells transfected with FLAG-mini-WT or FLAG-mini-FANSHY showed that significantly more FLAG-mini-FANSHY is secreted into the medium (lanes 5 and 6) compared to FLAG-mini-WT (lanes 3 and 4) (representative blots and bar graphs from quantifications of five independent experiments). Empty vector without any mini-receptor construct (pcDNA) was included as a negative control.

C) Quantification of the Western Blot of B).

D-E) FACS analysis comparing the cell surface distribution of FLAG-mini-WT and FLAG-mini-FANSHY in transfected cells. For example, whereas the number of positive cells as defined reaching a predefined threshold of the antibody signal was 321 and 347 for two experiments for FLAG-mini-WT (upper panels in D and E, respectively), this number was increased to 592 and 509 for cells transfected with FLAG-mini-FANSHY (lower panels in D and E, respectively). Moreover, the signal was lower for cells expressing FLAG-mini-WT (Mean signals 4.635.522 and 4.736.355; upper panels in D and E respectively) in comparison to FLAG-mini-FANSHY (Mean signals 5.286.295 and 5.285.501; lower panels in D and E, respectively).

F) Immunocytochemistry detection of FLAG-mini-WT and FLAG-mini-FANSHY at the cell surface (stained in the absence of detergent/TritonX-100) of transfected cells. Clearly, there is a stronger accumulation of FLAG-mini-FANSHY at the cell surface compared to FLAG-mini-WT (arrows).

FIG. 11: SORL1 dimerization by its 3Fn domains.

A) Schematic representations showing domain architectures of the SORLA-WT protein with all its natural domains (VPS10p, YWTD, CR domains, FnIII domains), variants of the 3Fn minireceptor with different tags (mini-myc and FLAG-mini, respectively) and the soluble 3Fn minireceptors used in Example 8.

Vps10p, Vacuolar Protein Sorting 10 Protein; YWTD, Tyrosine Tryptophan Threonine Aspartate; CR, complement-type repeats; 3Fn, Fibronectin-type-III domain; TM, transmembrane; 10CC, 10 conserved cysteines; EGF, Epidermal Growth Factor. B) Co-immunoprecipitation of lysates from HEK293 cells transiently transfected with the FLAG-tagged minireceptor and SORL1-WT using FLAG antibody. Proteins present in the eluates were analyzed by WB using a polyclonal SORL1 antibody (sol-SORLA).

C) Co-immunoprecipitation analysis of lysates from HEK293 cells transiently transfected with FLAG-tagged minireceptor and Myc-tagged minireceptor. Proteins were precipitated using a FLAG antibody and detected by WB using a myc antibody.

D) WB analysis of lysates and media from CHO cells transiently transfected with soluble 3Fn minireceptor (without the transmembrane or tail domain) treated with either 0 or 5% (+β-Mercaptoethanol) reducing agent. An anti-FLAG antibody is used for detection of the soluble 3Fn minireceptor constructs containing an N-terminal 3× FLAG-tag.

FIG. 12: Cellular analysis of SORLA dimerization in endosomes

A) Schematics showing the principle behind the proximity ligation assay (PLA). When the two SORL1 constructs with a C-terminal FLAG-tag (SORL1-FLAG) or GFP-tag (SORL1-GFP) interact (i.e. dimerize) a red PLA signal develops in a number of steps.

B) Representative confocal microscopy images of HEK293 cells transiently co-transfected with SORL1-GFP (*) and SORL1-FLAG. Cells were immunostained for SorLA using FLAG antibody (arrow) and nuclear counterstained with DAPI (blue). Scale bars 5 μm. We tested whether the two different tags affected the localization of the tagged receptor forms using double transfected HEK293 cells and determination of their degree of co-localization. From this experiment we observed no indication that the tags lead to differences in cellular localization as they showed a strong overlap and Mander's coefficient of 0.71.

C-D) Next, we performed control experiments for the PLA setup but leaving out either the anti-FLAG (C) or anti-GFP (D) that in both settings abolished formation of the PLA signal.

E) Representative images from PLA analysis of the interaction between SORL1-GFP (*) and SORL1-FLAG in transiently transfected HEK293 cells. PLA signals are represented by dots (see arrows). Scale bars 5 μm. Quantification of colocalization between GFP (*) and PLA (arrows) signals using Mander's Correlation Coefficient. In the presence of both antibodies, we observed a strong PLA signal confirming that two full-length SORLA proteins can form a dimer within cells. However, when we determined the degree of co-localization between the PLA signal (representing SORLA dimers) and SORLA-GFP (representing both monomers and dimers), we noticed a clear lack of co-localization for several PLA signals and Mander's coefficient confirmed an overlap of only 0.35, suggesting that only a fraction of the cellular pool of SORLA protein exist as dimers.

F) Co-staining for the PLA signal representing dimers and the VPS35 subunit of the retromer core showed a high degree of co-localization with Mander's coefficient of 0.70, confirming that the majority of SORLA dimers are located together with retromer.

FIG. 13: The intracellular localization of the SORL1 dimers is within endosomes.

(A) Schematic depiction of SORL1 constructs containing C-terminal fusions of either the N-terminal (SORL1-V1) or C-terminal (SORL1-V2) fragment of the venus fluorescent protein. Upon interaction between the two SORL1 molecules the venus fragments get into close proximity enabling them to form a fluorescent beta-barrel structure

B) Retromer interacts specifically with SORL1 dimers. HEK293 cells were transfected with an empty vector SORL1-GFP, SORL1-V1, SORL1-V2, both SORL1-V1 and SORL1-V2 (expressing the various SORLA variants), or SORL1-WT, followed by immunoprecipitation with GFP-trap. Protein in the eluates were analyzed by WB using the indicated antibodies for detection of SORLA and VPS26B.

C) Representative immunofluorescence images of BiFC signals (*) from HEK293 cells transiently transfected with either SORL1-V1 (upper row), SORL1-V2 (middle row) or co-transfected with both SORL1-V1 and SORL1-V2 (lower row). The cells were counter-immunostained with a SORL1 antibody (arrow) and nuclei were stained with DAPI. Scale bars 5 μm.

D) Quantification of colocalization of venus signal with total SORL1 using Mander's Correlation Coefficient.

### DETAILED DESCRIPTION

### Definitions

As used herein, the singular forms “a”, “an” and “the” include plural referents unless the context clearly states otherwise.

The term “some embodiments” can include one, or more than one embodiment.

The use of the word “a” or “an” when used throughout the text or in conjunction with the term “comprising” in the claims and/or the specification may mean “one,” but it is also consistent with the meaning of “one or more,” “at least one,” and “one or more than one.” Thus, for example, reference to “mini-receptor” includes a plurality of such mini-receptors, such as one or more mini-receptors, at least one mini-receptor, or two or more mini-receptors.

The term “construct” as used herein refers to a polynucleotide construct, for example as described in the Examples below or in the claims. For example, this construct refers to the SORL1 mini-gene, encoding the SORLA mini-receptor.

The term “sorLA” as used herein is synonymous to the terms SORLA, Sortilin-related receptor, sortilin related receptor 1, Low-density lipoprotein receptor relative with 11 ligand-binding repeats, LDLR relative with 11 ligand-binding repeats, LR11, SorLA-1, Sorting protein-related receptor containing LDLR class A repeats and gp250. Human sorLA is annotated in UniProt under the accession number Q92673.

The term “wildtype” (WT) is generally understood as an unmodified protein or protein fragment compared to a protein or protein fragment where a modification has been introduced.

The term “wildtype” (WT) for example used in Examples 1 and 2 is understood to describe a naturally occurring protein, i.e. naturally occurring sorLA, and may be understood as full-length sorLA.

The term “wildtype” (WT) as used in some instances in Example 8 is understood to describe non-mutated variant of the minireceptor (FLAG-mini-WT=SORLA-3Fn mini-receptor) in comparison to a mutant variant of the mini-receptor with substitution of the FANSHY motif by Alanine residues (FLAG-mini-FANSHY).

The understanding of the term “wildtype” is further clarified in the respective Examples 1-8 below.

The protein encoded by SORL1 is called SORLA, also known as Sortilin-related receptor, sortilin related receptor 1, Low-density lipoprotein receptor relative with 11 ligand-binding repeats, LDLR relative with 11 ligand-binding repeats, LR11, SorLA-1,

Sorting protein-related receptor containing LDLR class A repeats, gp250. Human SORLA is, for example, annotated under the accession number Q92673 at the UniProt database (UniProtKB-Q92673, SORL_HUMAN). SORLA is a type-1 membrane protein that is, for example, expressed in neurons. SORLA is known to play roles in endocytosis and sorting.

The by SORL1 encoded protein SORLA comprises
a signal peptide (cleaved off during maturation of the protein; encoded by exon 1),
a propeptide (may be proteolytically processed during maturation of the protein, encoded by exon 1),
a Vps10p domain (also referred to as Vps10p-β-propeller (encoded by exons 2-13)
a 10CC domain (encoded by exons 14-16)
a YWTD domain (also referred to as YWTD-β-propeller (encoded by exons 17-21)
an epidermal growth factor (EGF) domain (encoded by exon 22
a CR cluster domain (encoded by exons 23-33)
a 3Fn cluster, also referred to as 3Fn cassette, 3Fn repeats, 3Fn domain (encoded by exons 34-46), also referred to as six Fibronectin type III (FNIII or FN3) repeats
a transmembrane domain and a cytoplasmic tail domain (encoded by exons 47-48)

Throughout this disclosure, the term 3Fn may also be expressed as FNIII or FN3 (Fn=fibronectin).

The terms homology, identity and similarity, with respect to a polynucleotide (or polypeptide), as defined herein are used interchangeably and refer to the percentage of nucleic acids (or amino acids) in the candidate sequence that are, homolog, identical or similar, respectively, to the residues of a corresponding native nucleic acids (or amino acids), after aligning the sequences and introducing gaps, if necessary, to achieve the maximum percent identity/similarity, and considering any conservative substitutions according to the NCIUB rules (hftp://www.chem.qmul.ac.uk/iubmb/misc/naseq.html; NC-IUB, Eur J Biochem (1985)) as part of the sequence identity. In particular, the percentage of similarity refers to the percentage of residues conserved with similar physiochemical properties. Neither 5′ or 3′ extensions nor insertions (for nucleic acids) or N′ or C′ extensions nor insertions (for polypeptides) result in a reduction of identity or similarity. Methods and computer programs for the alignments are well known in the art. Generally, a given similarity between two sequences implies that the identity between these sequences is at least equal to the similarity; for example, if two sequences are 80% similar to one another, they cannot be less than 80% identical to one another—but could be sharing 90% identity.

As defined herein the term “at least 70% homology, similarity or identity” means at least 70%, at least 75%, at least 80%, at least 85%, at least 90%, at least 95%, at least 98% or at least 99% homology, similarity or identity throughout the present disclosure.

The terms “identity” and “homology” may be understood interchangeably herein.

### SORLA Mini-Receptor

The SORLA mini-receptor described herein comprises a cytoplasmic tail, a transmembrane region and a part of the luminal region, namely the FNIII cassette domain of SORLA.

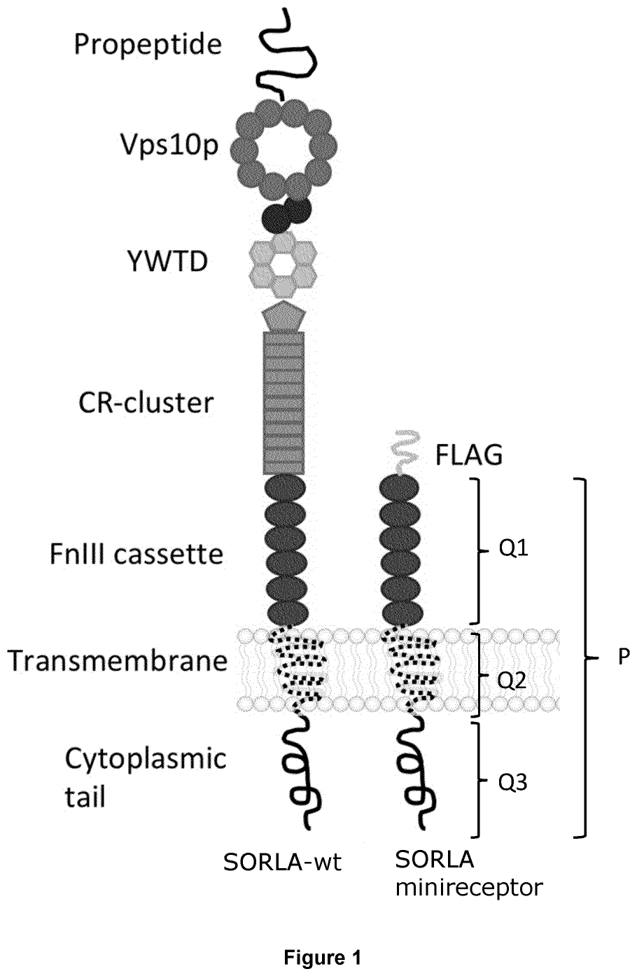

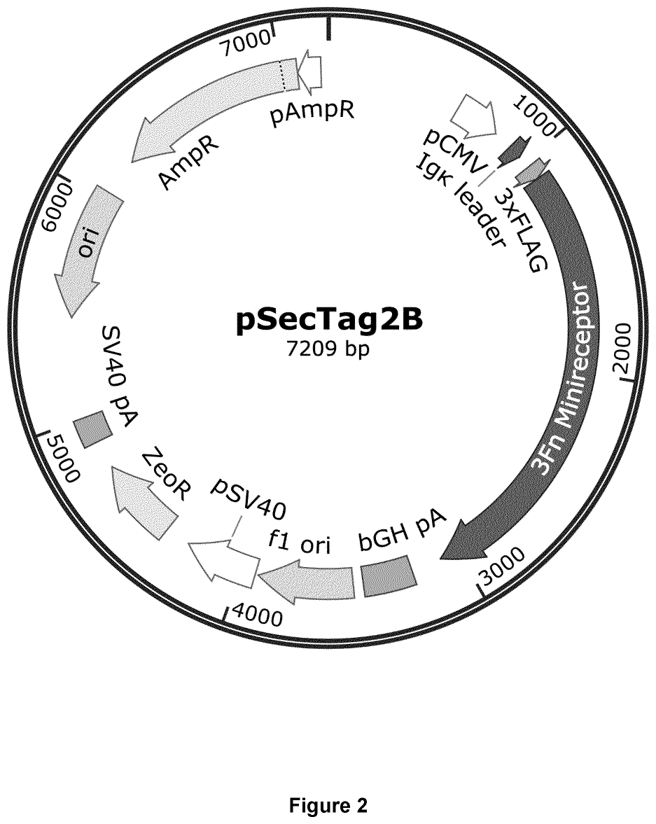

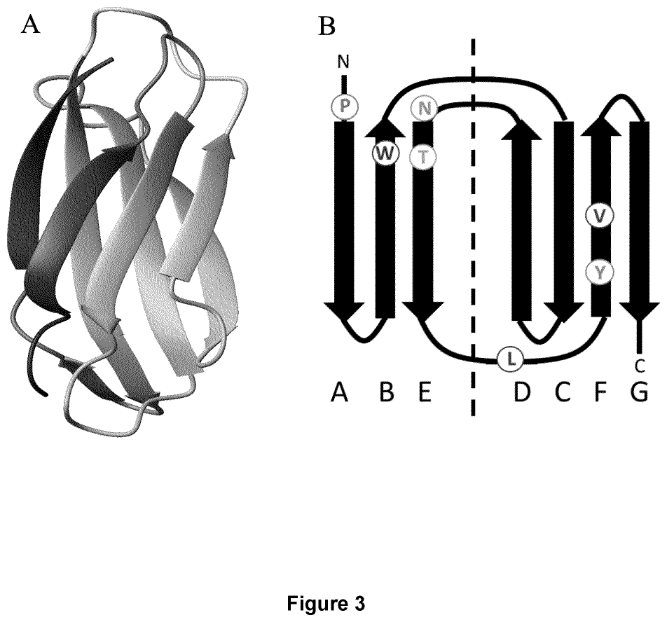

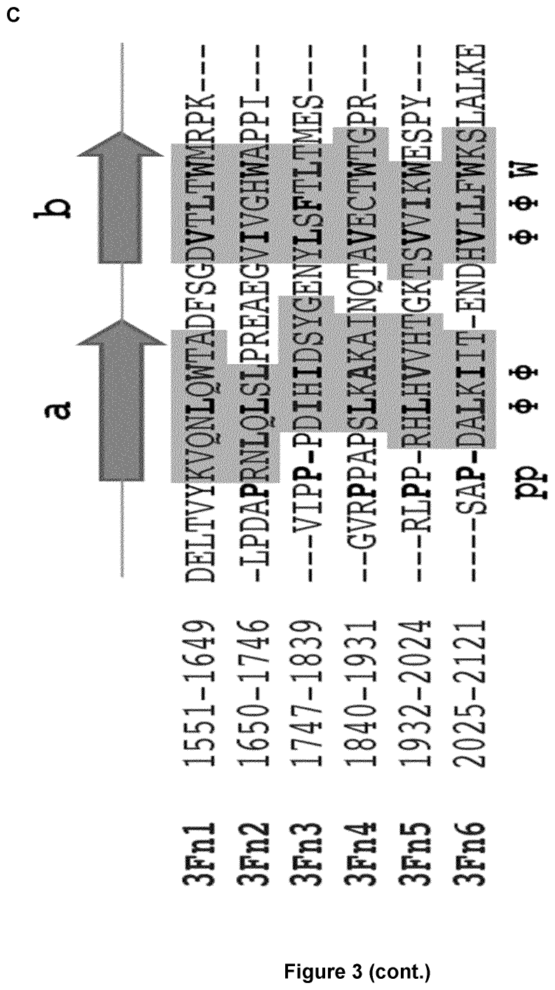

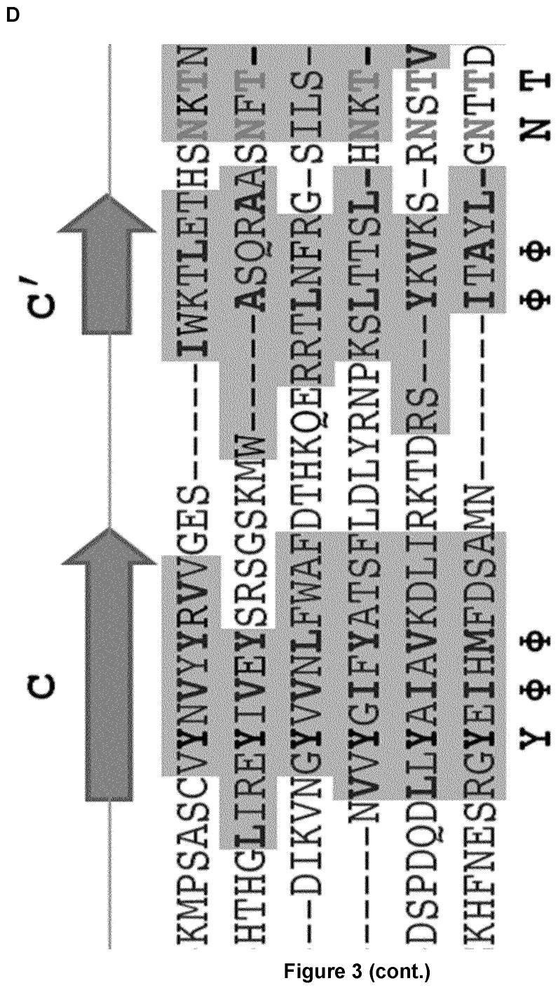

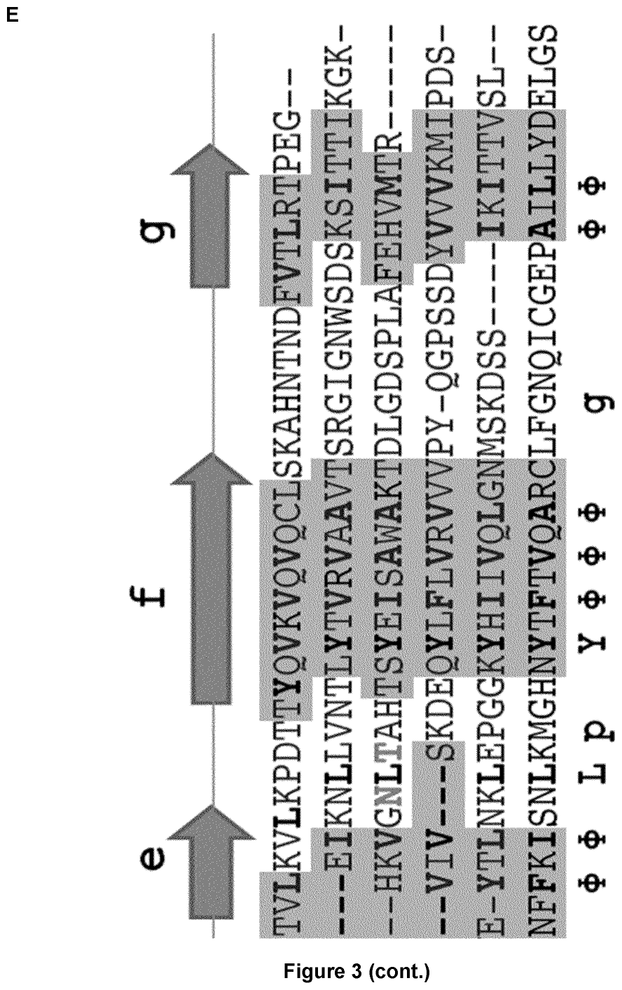

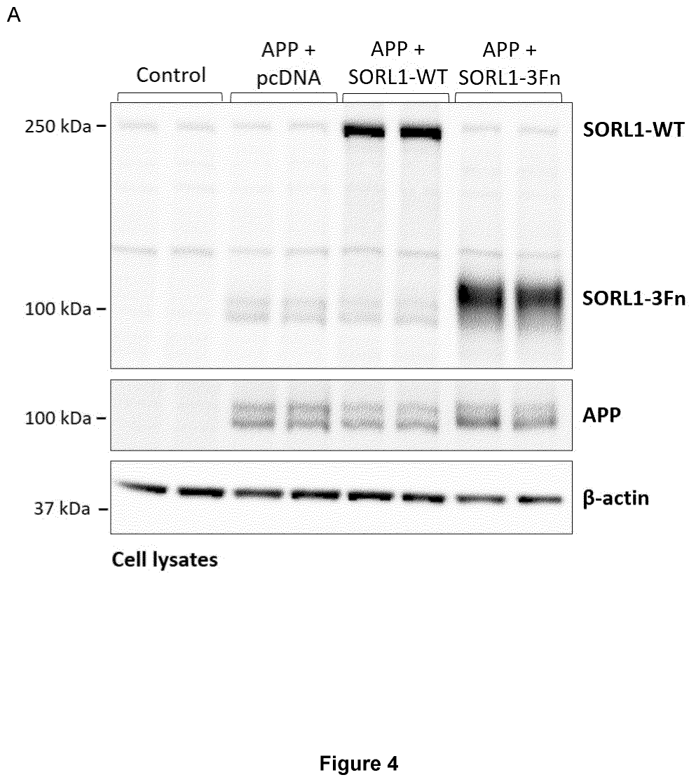

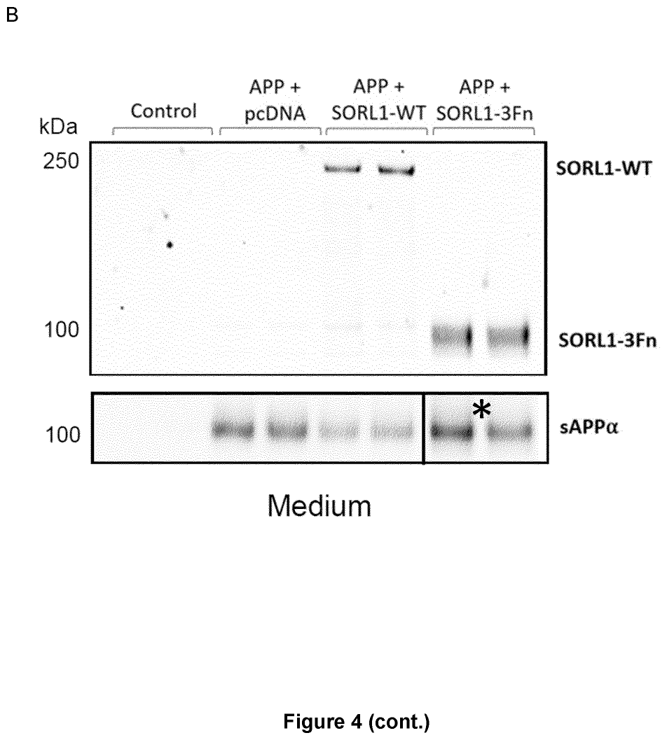

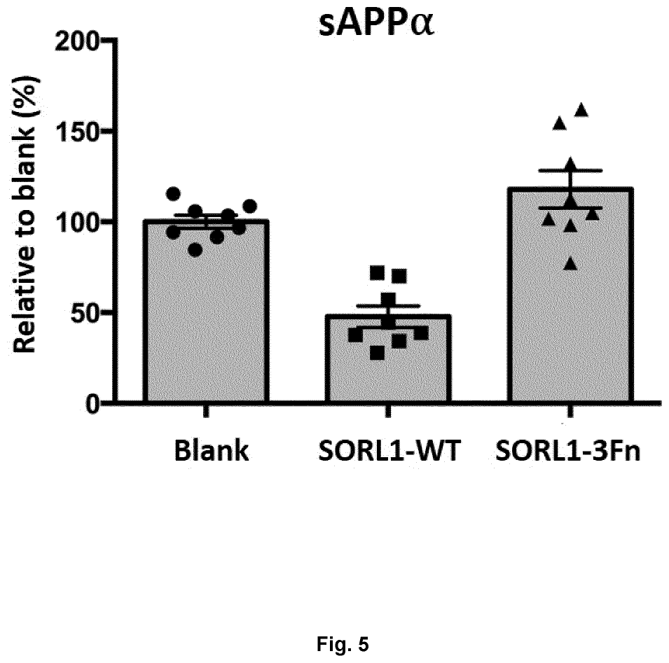

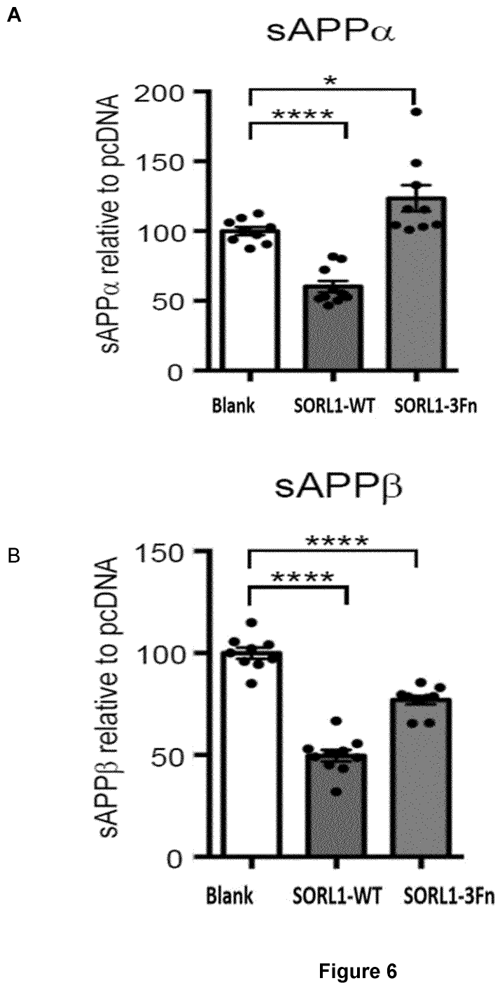

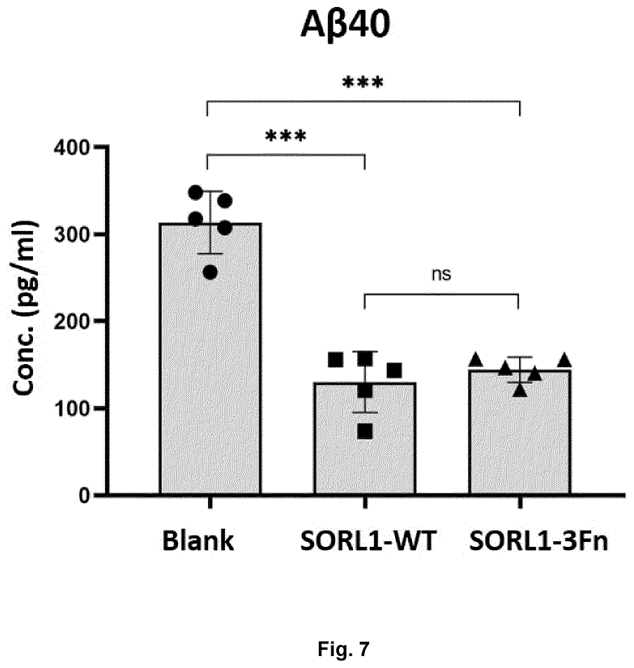

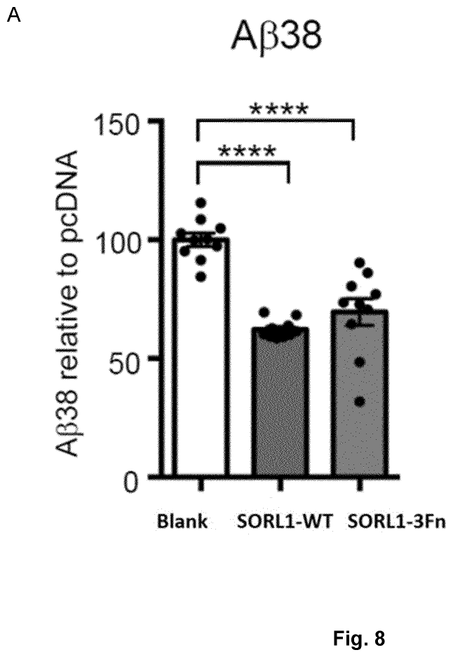

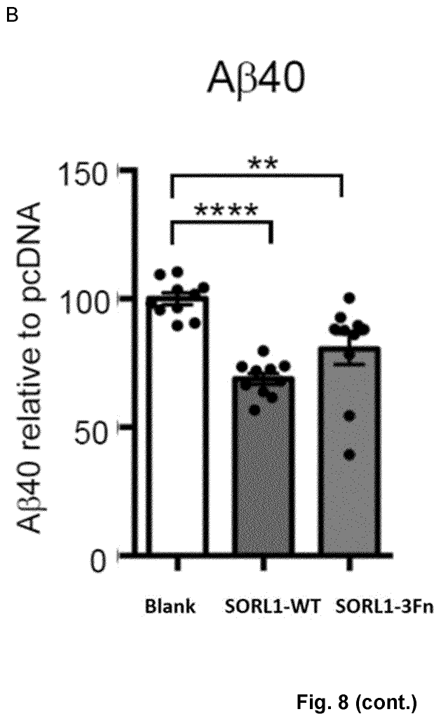

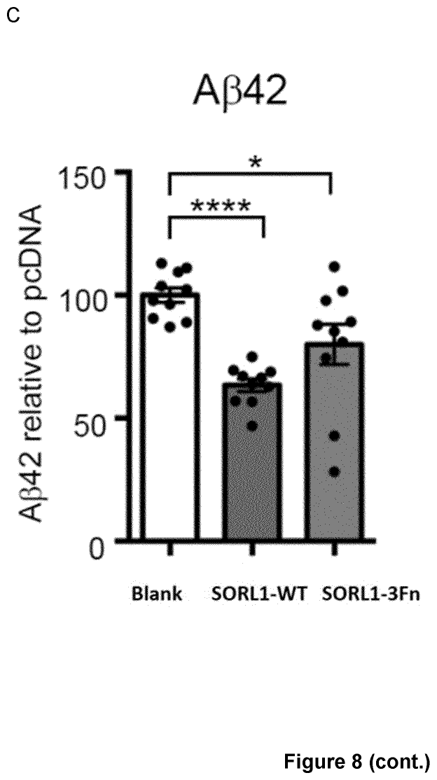

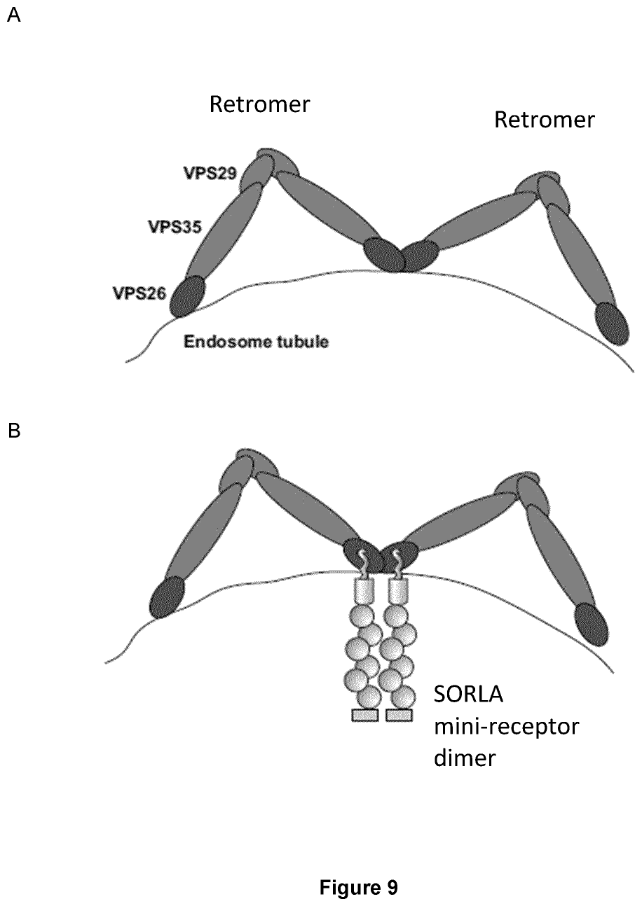

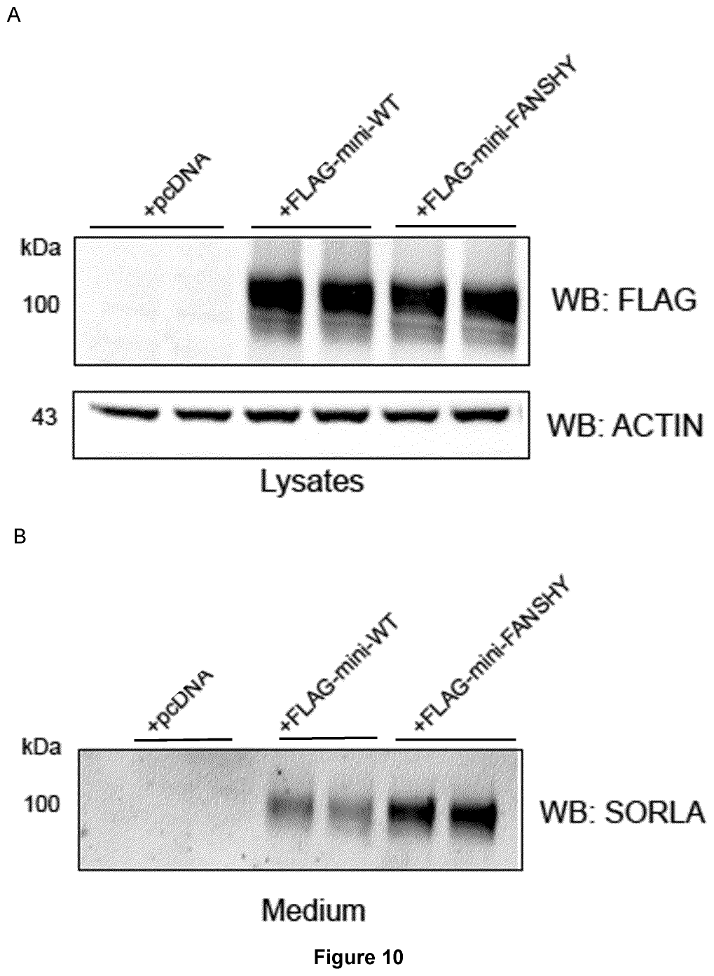

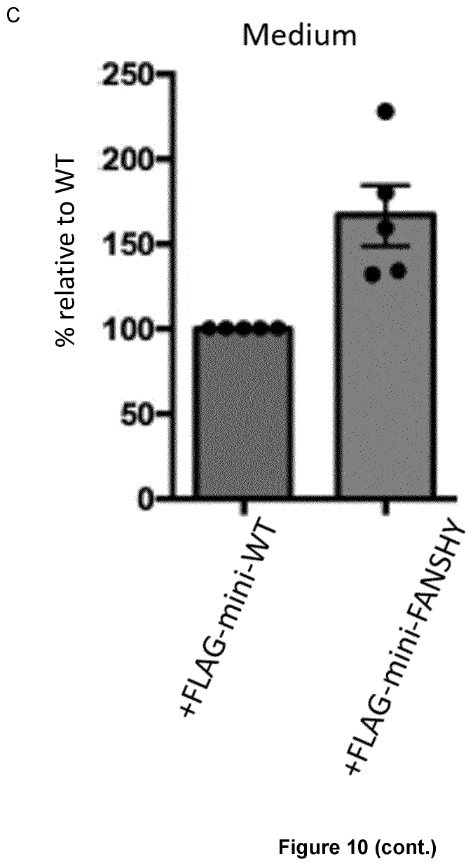

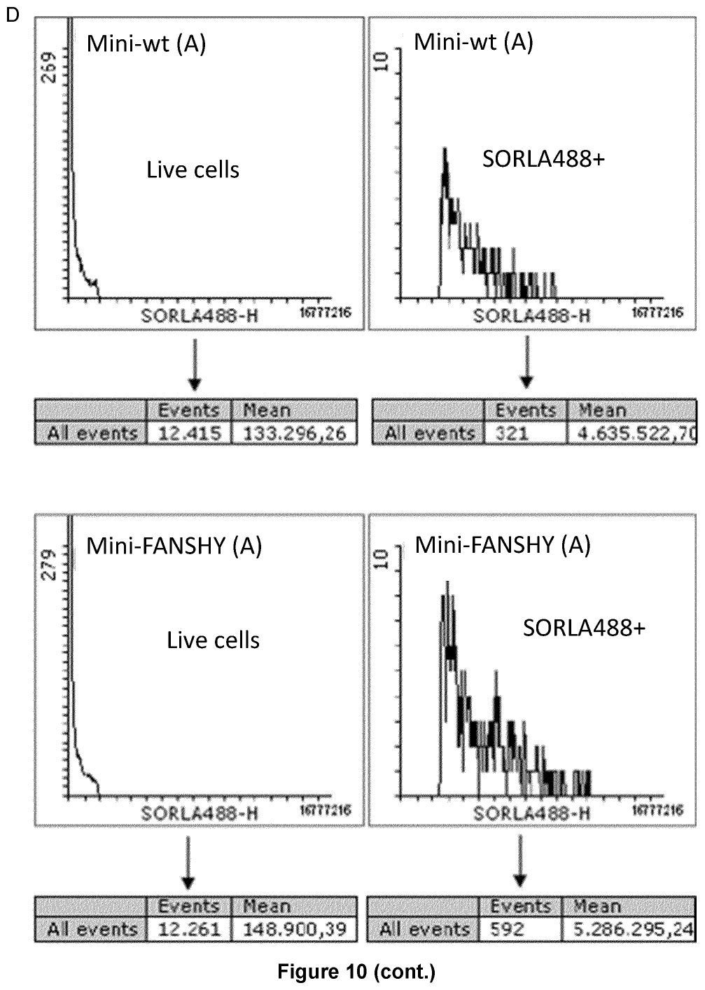

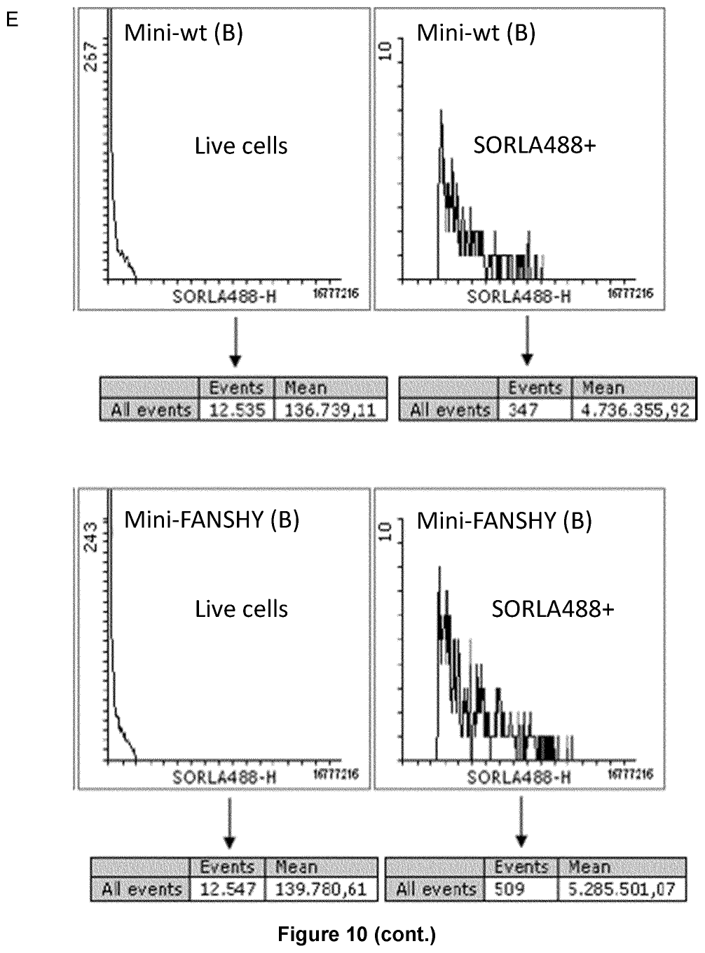

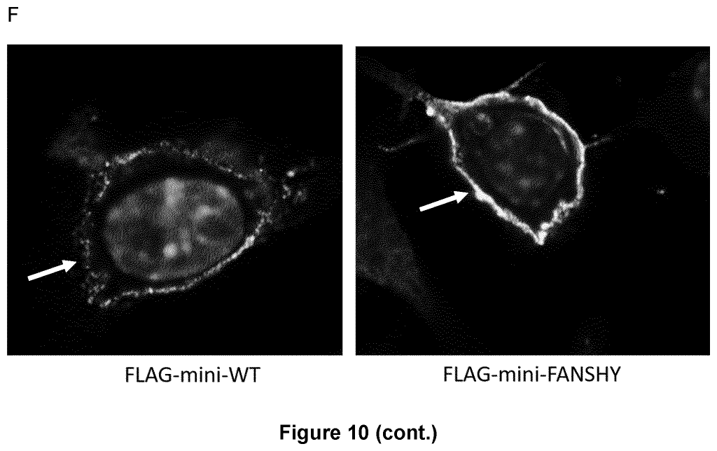

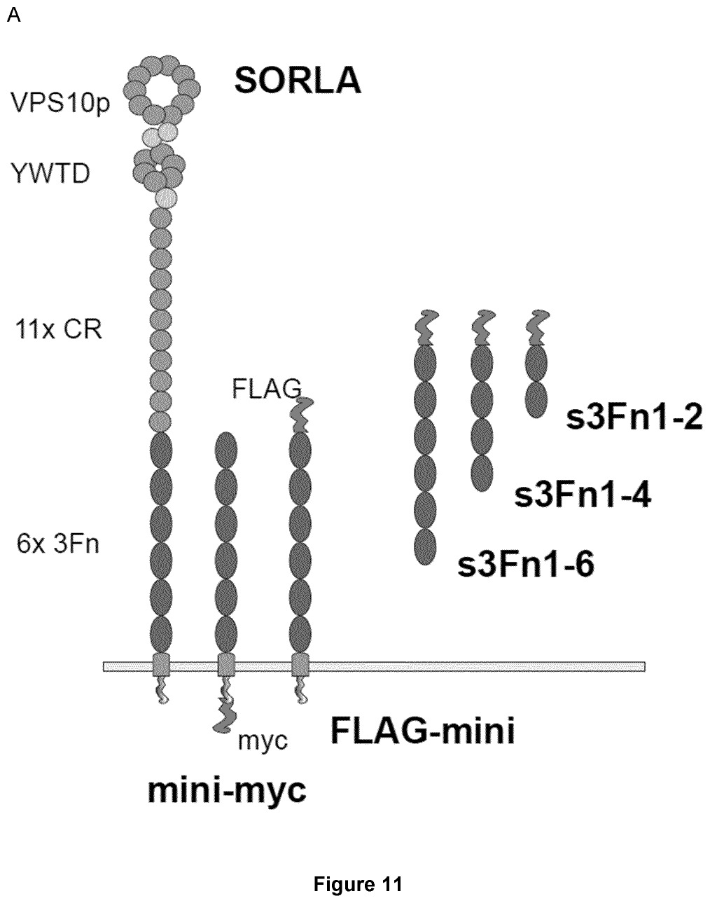

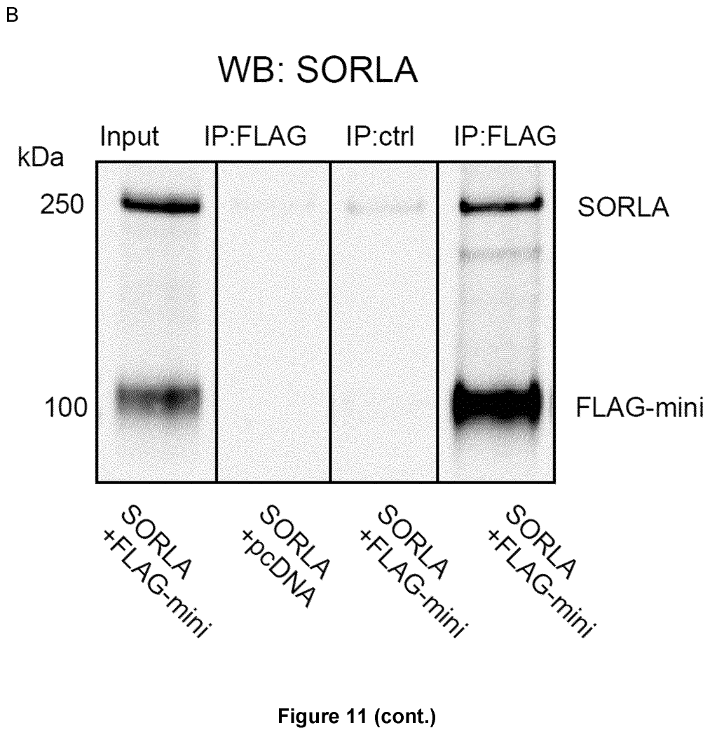

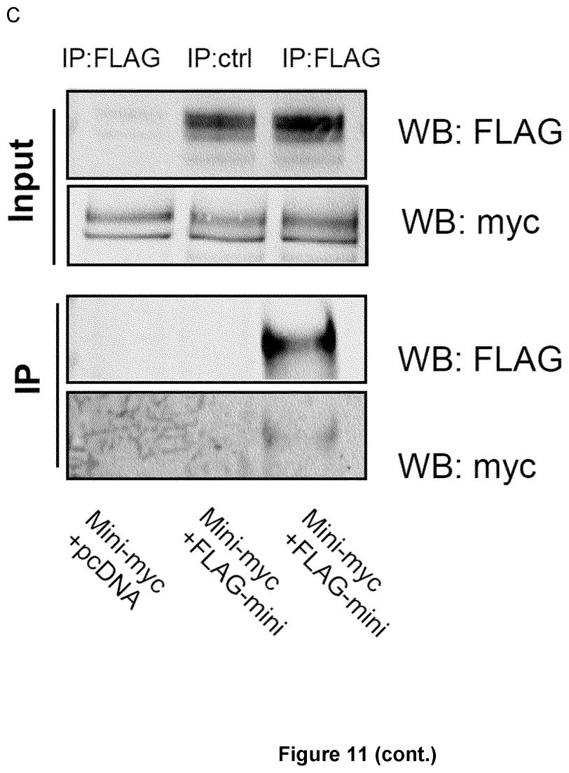

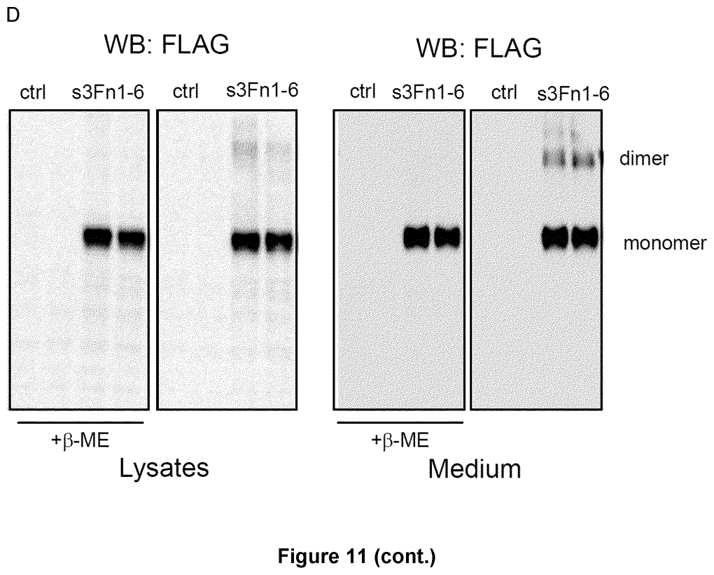

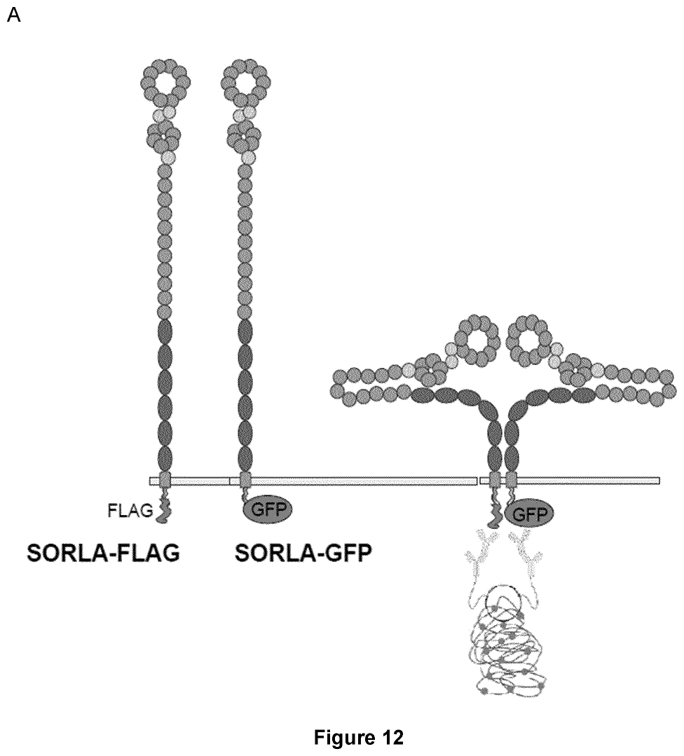

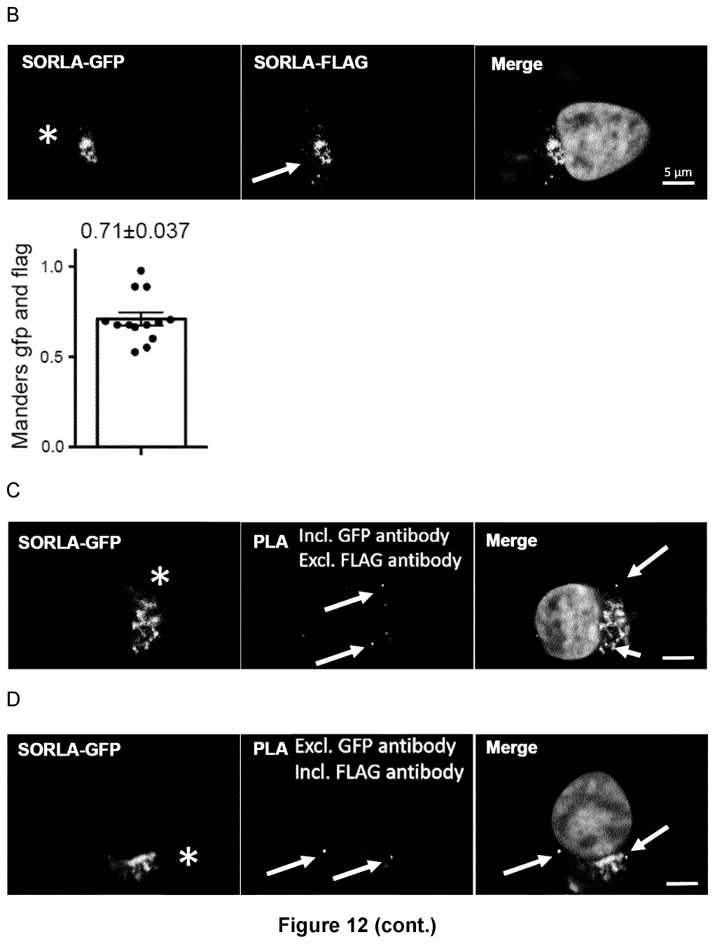

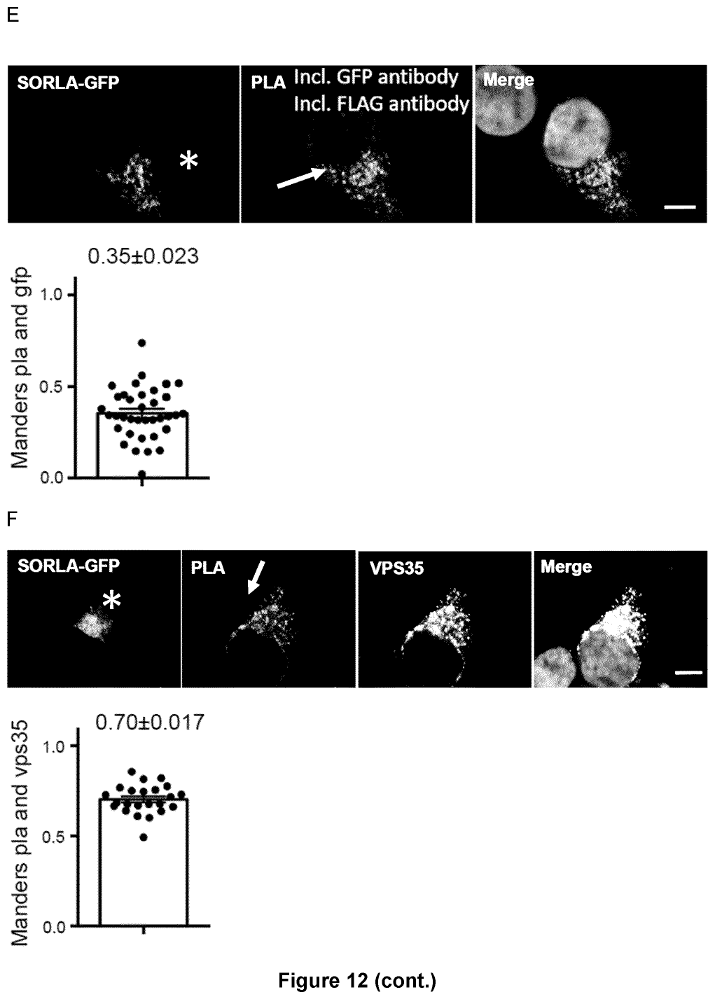

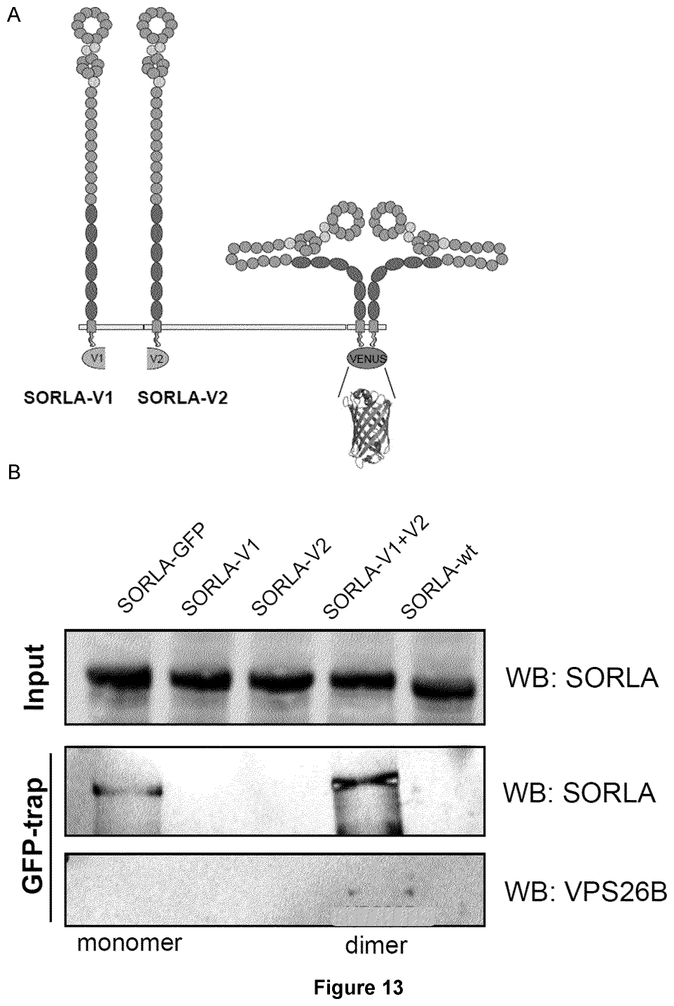

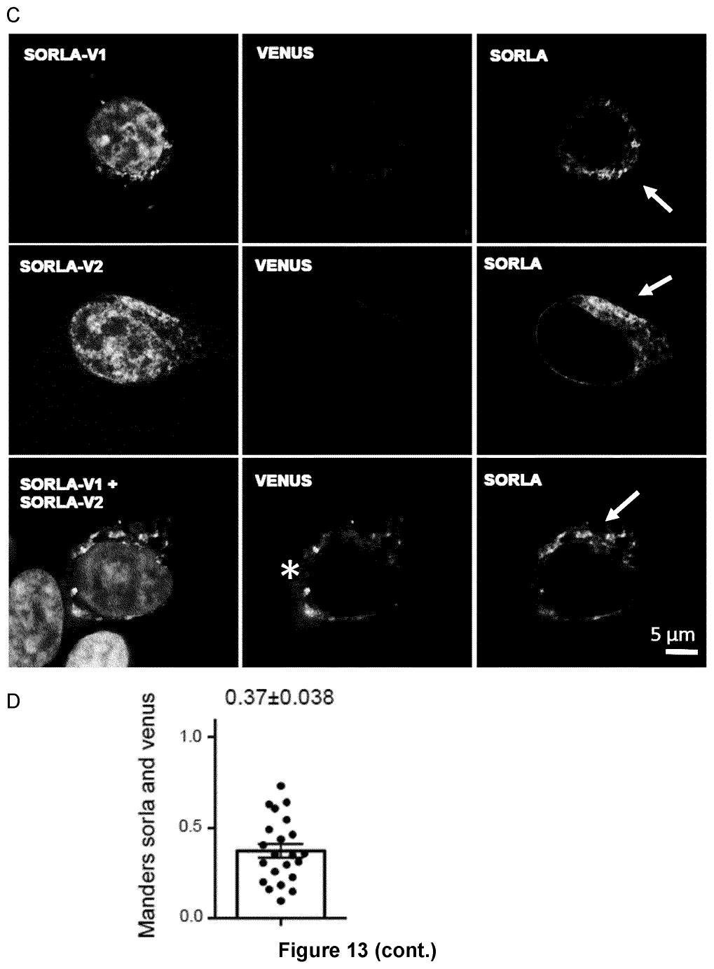

As depicted in FIG. 1, the mini-receptor comprises or consists of a polypeptide Q1 comprising one or more FnIII domains of SORLA (for example all 6 FnII domains), a polypeptide Q2 comprising or consisting of a transmembrane domain of SORLA, and a polypeptide Q3 comprising or consisting of a cytoplasmic tail domain of SORLA, which together may be referred to as polypeptide P.

Optionally, the mini-receptor may comprise a tag, for example a FLAG-tag.

The FNIII cassette, being in the luminal region and such interacting with other luminal proteins, is important for the functional properties of the SORLA mini-receptor. Thus, the SORLA mini-receptor may be referred to as the SORLA-3Fn mini-receptor (wherein 3FN is an abbreviation for FNIII cassette), as for example in Examples 1 to 6. The respective nucleotide sequence for expressing the SORLA mini-receptor may be referred to as SORL1-3Fn and may be referred to as the SORL1 mini-gene. Further, throughout the text and in relation to the present disclosure, SORLA mini-receptor may be referred to as mini-receptor only. In Examples 1 to 6, the denotation SORL1-WT or SorLA-wt refers to the full-length SORL1 nucleotide sequence or to the full-length SorLA protein. As such, the mini-receptor or mini-gene is compared to the full-length receptor or gene.

Alternatively, above described SORLA mini-receptor may, in the present disclosure, also be referred to as FLAG-mini-WT, as for example in Examples 7 to 8. In Examples 7 to 8, the established mini-receptor or mini-receptors (denoted FLAG-mini-WT) are compared to a mutated mini-receptor variant (denoted FLAG-mini-FANSHY), where mutations have been introduced to the FANSHY motif in the cytoplasmic tail.

In one aspect, the present invention relates to a polynucleotide construct encoding upon expression a polypeptide P, wherein the polynucleotide comprises or consists of
a. a first polynucleotide encoding upon expression a first polypeptide Q1 comprising or consisting of one or more amino acid sequences selected from the group consisting of SEQ ID NO: 11, SEQ ID NO: 12, SEQ ID NO: 13, SEQ ID NO: 14, SEQ ID NO: 15 and SEQ ID NO: 16, or a biologically active sequence variant thereof wherein the variant is at least 80%, such as at least 90%, such as at least 95%, such as at least 99% identical to said amino acid sequence of SEQ ID NO: 11, SEQ ID NO: 12, SEQ ID NO: 13, SEQ ID NO: 14, SEQ ID NO: 15 or SEQ ID NO: 16; and
b. a second polynucleotide encoding upon expression a second polypeptide Q2 comprising or consisting of the amino acid sequence of SEQ ID NO: 17, or a sequence which is at least 80% identical to SEQ ID: 17, such as at least 90%, such as at least 95%, such as at least 99%; and
c. a third polynucleotide encoding upon expression a third polypeptide Q3 comprising or consisting of the amino acid sequence of SEQ ID NO: 18, or a sequence which is at least 70% identical to SEQ ID: 18, such as at least 75%, such as at least 80%, such as at least 90%, such as at least 95%, such as at least 99%;
wherein the polypeptide P comprises no more than 700 amino acids.

The person skilled in the art will appreciate that a number of nucleotides may be added upstream and/or downstream (at the 5′ end of the polynucleotide and/or at the 3′ end of the polynucleotide, respectively) of the first polynucleotide, the second polynucleotide and/or the third nucleotide. This may be done to optimize the construct, for example to optimize transcription and/or translation, packaging efficiency or properties of the encoded polynucleotide (i.e. polynucleotide P of the invention). As the polypeptide P of the invention comprises no more than 700 amino acids, the amount of additional nucleotides in the polynucleotide construct is limited to a total number of 2100 nucleotides.

In some embodiments of the present disclosure, the first polynucleotide, the second polynucleotide and/or the third polynucleotide comprise further nucleotides at the 3′ end and/or at the 5′ end, such as 1 further nucleotide, such as 2 further nucleotides, such as 3 further nucleotides, such as 4 further nucleotides, such as 5 further nucleotides, such as 6 further nucleotides, such as 7 further nucleotides, such as 8 further nucleotides, such as 9 further nucleotides, such as 10 further nucleotides, such as 11 further nucleotides, such as 12 further nucleotides, such as 13 further nucleotides, such as 14 further nucleotides, such as 15 further nucleotides, such as 16 further nucleotides, such as 17 further nucleotides, such as 18 further nucleotides, such as 19 further nucleotides, such as 20 further nucleotides, such as 21 further nucleotides, such as 22 further nucleotides, such as 23 further nucleotides, such as 24 further nucleotides, such as 25 further nucleotides, such as 26 further nucleotides, such as 27 further nucleotides, such as 28 further nucleotides, such as 29 further nucleotides, such as 30 further nucleotides, such as 31 further nucleotides, such as 32 further nucleotides, such as 33 further nucleotides.

The one or more further nucleotide may be any known type of nucleotide.

In some embodiments of the present invention the polynucleotide construct encodes upon expression a fusion protein comprising a first polypeptide Q1, a second polypeptide Q2 and a third polypeptide Q3.

In an alternative aspect, the present invention relates to a polynucleotide construct comprising
a. a first polynucleotide encoding upon expression a polypeptide P, comprising or consisting of
   a first polypeptide Q1 comprising or consisting of one or more amino acid sequences selected from the group consisting of SEQ ID NO: 11, SEQ ID NO: 12, SEQ ID NO: 13, SEQ ID NO: 14, SEQ ID NO: 15 and SEQ ID NO: 16, or a biologically active sequence variant thereof wherein the variant is at least 80%, such as at least 90%, such as at least 95%, such as at least 99% identical to said amino acid sequence of SEQ ID NO: 11, SEQ ID NO: 12, SEQ ID NO: 13, SEQ ID NO: 14, SEQ ID NO: 15 and SEQ ID NO: 16; and
b. a second polynucleotide encoding upon expression a second polypeptide Q2 comprising or consisting of the amino acid sequence of SEQ ID NO: 17, or a sequence which is at least 80% identical to SEQ ID: 17, such as at least 90%, such as at least 95%, such as at least 99%; and
c. a third polynucleotide encoding upon expression a third polypeptide Q3 comprising or consisting of the amino acid sequence of SEQ ID NO: 18, or a sequence which is at least 70% identical to SEQ ID: 18, such as at least 75%, such as at least 80%, such as at least 90%, such as at least 95%, such as at least 99%;
wherein the polypeptide P comprises no more than 700 amino acids.

In some embodiments of the present disclosure, P comprises or consists of
a. a FnIII cassette of SorLA comprising or consisting of amino acid sequences of SEQ ID NO: 11, SEQ ID NO: 12, SEQ ID NO: 13, SEQ ID NO: 14, SEQ ID NO: 15 and SEQ ID NO: 16, or an FnIII domain selected from FnIII-1, FnIII-2, FnIII-3, FnIII-4, FnIII-5 or FnIII-6, corresponding to the amino acid sequences of SEQ ID NO: 11, SEQ ID NO: 12, SEQ ID NO: 13, SEQ ID NO: 14, SEQ ID NO: 15 or SEQ ID NO: 16, or any combination thereof; and
b. a transmembrane domain of SorLA comprising or consisting of amino acid sequences of SEQ ID NO: 17, and
c. a cytoplasmic tail domain of SorLA comprising or consisting of amino acid sequences of SEQ ID NO: 18.

In some embodiments of the present disclosure, P is no longer than 685 amino acids.

In some cases, it may be beneficial to express the mini-receptor construct of the invention comprising a tag. The person skilled in the art will be able to select the most appropriate tag, as well as the localization of the tag. A tag may be, for example, a FLAG tag (e.g. referred to as FLAG-mini in Examples 7 to 8) or a myc tag (e.g. referred to as mini-myc in Examples 7 to 8) or a GFP-tag or a venus fluorescent protein-tag (e.g. used in Example 8).

The tag may, for example, be localized N-terminal or C-terminal on the construct. In some instances, it may be beneficial to localize the tag on the N-terminal of the construct so as to leave the C-terminal unmodified and thus not potentially affecting any binding with other molecules binding to the C-terminal region of the construct, for example retromer binding to the cytoplasmic tail.

Usage of a tag may be beneficial in tracking the construct in-vitro or in-vivo to confirm the localisation of the construct. In other cases, for example in a clinical setting when transfection patient cells with the construct, e.g. Adeno-associated virus (AAV)-mediated, it may be beneficial to omit any tag with the aim of not introducing any non-essential protein parts that may potentially trigger an immunogenic response.

In some preferred embodiments of the disclosure, the mini-receptor does not comprise any tag.

In some embodiments of the present disclosure, P further comprises the amino acid sequence of SEQ ID NO: 21.

In some embodiments of the present disclosure, P comprises or consists of the amino acid sequences of SEQ ID NO: 11, SEQ ID NO: 12, SEQ ID NO: 13, SEQ ID NO: 14, SEQ ID NO: 15, SEQ ID NO: 16, SEQ ID NO: 17 and SEQ ID NO: 18.

In some embodiments of the present disclosure, P comprises or consists of the amino acid sequence of SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18 and SEQ ID NO: 21.

In some embodiments of the present disclosure, and in contrast to full-length SORLA protein, the construct does not contain the CR-cluster domain, the YWTD domain, the Vps10p domain and/or the propeptide.

In some embodiments of the present disclosure, and in contrast to full-length SORLA protein the construct does not contain the CR-cluster domain.

In some embodiments of the present disclosure, and in contrast to full-length SORLA protein, the construct does not contain the YWTD domain.

In some embodiments of the present disclosure, and in contrast to full-length SORLA protein, the construct does not contain the Vps10p domain.

In some embodiments of the present disclosure, and in contrast to full-length SORLA protein, the construct does not contain the propeptide.

In another aspect, the present invention relates to a polypeptide P, comprising or consisting of
a. a first polypeptide Q1 comprising or consisting of one or more amino acid sequences selected from the group consisting of SEQ ID NO: 11, SEQ ID NO: 12, SEQ ID NO: 13, SEQ ID NO: 14, SEQ ID NO: 15 and SEQ ID NO: 16, or a biologically active sequence variant thereof wherein the variant is at least 80%, such as at least 90%, such as at least 95%, such as at least 99% identical to said amino acid sequence of SEQ ID NO: 11, SEQ ID NO: 12, SEQ ID NO: 13, SEQ ID NO: 14, SEQ ID NO: 15 or SEQ ID NO: 16; and
b. a second polypeptide Q2 comprising or consisting of the amino acid sequence of SEQ ID NO: 17, or a sequence which is at least 80% identical to SEQ ID: 17, such as at least 90%, such as at least 95%, such as at least 99%; and
c. a third polypeptide Q3 comprising or consisting of the amino acid sequence of SEQ ID NO: 18, or a sequence which is at least 70% identical to SEQ ID: 18, such as at least 75%, such as at least 80%, such as at least 90%, such as at least 95%, such as at least 99%;
   wherein the polypeptide P comprises no more than 700 amino acids.

In some embodiments of the present disclosure, the first polypeptide Q1, the second polypeptide Q2 and/or the third polypeptide Q3 comprise further amino acids, such as 1 further amino acid, such as 2 further amino acids, such as 3 further amino acids, such as 4 further amino acids, such as 5 further amino acids, such as 6 further amino acids, such as 7 further amino acids, such as 8 further amino acids, such as 9 further amino acids, such as 10 further amino acids, such as 11 further amino acids, such as 12 further amino acids, such as 13 further amino acids, such as 14 further amino acids, such as 15 further amino acids, such as 16 further amino acids, such as 17 further amino acids, such as 18 further amino acids, such as 19 further amino acids, such as 20 further amino acids, such as 21 further amino acids, such as 22 further amino acids, such as 23 further amino acids, such as 24 further amino acids, such as 25 further amino acids, such as 26 further amino acids, such as 27 further amino acids, such as 28 further amino acids, such as 29 further amino acids, such as 30 further amino acids, such as 31 further amino acids, such as 32 further amino acids, such as 33 further amino acids.

The one or more further amino acid may be any known type of amino acid.

In some embodiments of the present disclosure, the polypeptide P is a fusion protein of polypeptide Q1, Q2 and/or Q3.

In some embodiments of the present disclosure, there is a linker, comprising of further amino acids, between polypeptide Q1, polypeptide Q2 and polypeptide Q3.

In some embodiments of the present disclosure polypeptide P comprises or consists of
a. a FnIII cassette of SorLA comprising or consisting of amino acid sequences of SEQ ID NO: 11, SEQ ID NO: 12, SEQ ID NO: 13, SEQ ID NO: 14, SEQ ID NO: 15 and SEQ ID NO: 16, or
b. an FnIII domain selected from FnIII-1, FnIII-2, FnIII-3, FnIII-4, FnIII-5 or FnIII-6, corresponding to the amino acid sequences of SEQ ID NO: 11, SEQ ID NO: 12, SEQ ID NO: 13, SEQ ID NO: 14, SEQ ID NO: 15 or SEQ ID NO: 16, or any combination thereof; and
c. a transmembrane domain of SorLA comprising or consisting of amino acid sequences of SEQ ID NO: 17, and
d. a cytoplasmic tail domain of SorLA comprising or consisting of amino acid sequences of SEQ ID NO: 18.

In some embodiments of the present disclosure, P is no longer than 685 amino acids.

In some embodiments of the present disclosure polypeptide P further comprises the amino acid sequence of SEQ ID NO: 21.

In some embodiments of the present disclosure polypeptide P comprises or consists of the amino acid sequences of SEQ ID NO: 11, SEQ ID NO: 12, SEQ ID NO: 13, SEQ ID NO: 14, SEQ ID NO: 15, SEQ ID NO: 16, SEQ ID NO: 17 and SEQ ID NO: 18.

In some embodiments of the present disclosure polypeptide P comprises or consists of the amino acid sequence of SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18 and SEQ ID NO: 21.

In another aspect, the present invention relates to a vector comprising said polynucleotide construct and/or encoding said polypeptide P.

In some embodiments of the present disclosure, the vector is selected from the group comprising of viral vectors, plasmids, cosmids and artificial chromosomes.

In some embodiments of the present disclosure, the vector is a plasmid vector.

In some embodiments of the present disclosure, the vector is a viral vector.

In some embodiments of the present disclosure, the vector has a packaging capacity from 1 to 40 kb, for example from 1 to 30 kb, such as from 1 to 20 kb, for example from 1 to 15 kb, such as from 1 to 10, for example from 1 to 8 kb, such as from 2 to 7 kb, for example from 3 to 6 kb, such as from 4 to 5 kb.

In some embodiments of the present disclosure, the viral vector is selected from the group consisting of adeno-associated vector (AAV), lentiviral vector, adenoviral vector and retroviral vector.

In some embodiments of the present disclosure, the viral vector is an adeno associated vector (AAV).

In some embodiments of the present disclosure, the viral vector is an AAV9 vector.

In some preferred embodiments of the present disclosure, the viral vector is an AAV9 vector.

In some embodiments of the present disclosure, the vector is an adeno-associated vector (AAV) with a packing capacity of 4.7 kb.

In some embodiments of the present disclosure, the vector is an adeno-associated vector (AAV) with a packing capacity of less than 5 kb.

The person skilled in the art will appreciate that Adeno-associated virus (AAV) vectors have limited packaging capacity. Over-packaging is for example detrimental to expression or limited success of expression, and can lead to reduction in both viral titers and in vivo transduction. While efforts are undertaken to optimize AAV vector packaging, the herein disclosed invention offers a solution to the problem by establishing a SORL1 mini-gene, which can be packaged into an AAV vector, instead of the full-length SORL1 gene which is over 6000 nucleotides long. AAV vectors are generally regarded to have a packaging capacity between around 4700 nucleotides to 5000 nucleotides. Additionally, the SORLA mini-receptor, transcribed from the SORL1 mini-gene, has further beneficial properties, as described in the herein enclosed examples below.

Human SORLA is annotated in UniProt under the accession number Q92673 as a protein of 2214 amino acids. This corresponds to a minimum of 6642 nucleotides to express/encode the protein. Further upstream or downstream sequences may be needed for expression. As such, full-length SORL1 is too large to be packaged into an AAV vector. The mini-gene solves this problem.

In an alternative aspect, the present invention relates to an AAV vector comprising said polynucleotide construct.

In a further alternative aspect, the present invention relates to an AAV9 vector comprising said polynucleotide construct.

In a further aspect, the present invention relates to a cell comprising said construct and/or said polypeptide P and/or vector.

In some embodiments of the present disclosure, the cell is selected from the group consisting of eukaryotic cells, preferably mammalian cells, more preferably primate cells, more preferably human cells.

In some embodiments of the present disclosure, the cell is a mammalian cell.

In some embodiments of the present disclosure, the cell is a neural cell.

In a further aspect, the present invention relates to a composition comprising said construct, polypeptide P, vector and/or cell.

In some embodiments of the present disclosure the composition is a pharmaceutical composition.

In some embodiments of the present disclosure the composition further comprises a pharmaceutically acceptable carrier.

In a further aspect, the present invention relates to said construct, polypeptide P, vector, cell and/or composition for use in medicine.

In a further aspect, the present invention relates to said construct, polypeptide P, vector, cell and/or composition for use in treatment, prevention and/or alleviation of Alzheimer's Disease or a disease or disorder associated with Alzheimer's Disease.

In some embodiments of the present disclosure, the Alzheimer's disease is of a type selected from the group consisting of Early-Onset Alzheimer's Disease, Late-Onset Alzheimer's Disease and Familial Alzheimer's Disease.

The person skilled in the art will appreciate that the present invention can be used in the treatment of individuals at risk for developing AD. For example, an individual at risk for developing AD may be an individual having a relative being diagnosed or recognised as having AD.

In an alternative aspect, the present invention relates to said construct, polypeptide, vector, cell and/or composition for use in treatment and/or prevention Alzheimer's Disease or a disease or disorder associated with Alzheimer's Disease in an individual at risk of developing Alzheimer's Disease.

The person skilled in the art will appreciate that the herein disclosed invention can be used to prevent AD and/or treat AD and/or alleviate symptoms of AD in AD patients. AD is a devastative disease affecting the brain on a structural level. It may therefore be beneficial to provide the SORL1 mini-gene (expressing the SORLA mini-receptor, e.g. via an AAV-vector), at an early stage, e.g. prior to the onset of symptoms, or at an early AD stage, or prior to substantive structural brain remodeling. For example, the herein described approach would be applicable to treat family members of AD patients prior to disease development.

In some embodiments of the present disclosure, said construct, vector, cell and/or composition are administered to an individual prior to the onset of AD symptoms.

In some embodiments of the present disclosure, said construct, vector, cell and/or composition is administered to an individual prior to the onset of AD symptoms.

In some embodiments of the present disclosure, said construct, vector, cell and/or composition is administered to an individual when a family member is diagnosed with AD.

Alternatively, the herein disclosed invention would be applicable to treat patients with early symptoms.

In some embodiments of the present disclosure, said construct, vector, cell and/or composition is administered to an individual with early AD symptoms.

Alternatively, the herein disclosed invention would be applicable to treat patients at various disease stages of AD.

In some embodiments of the present disclosure, said construct, vector, cell and/or composition is administered to an individual with a varying degree of AD symptoms.

Further, the herein disclosed invention would, due to its nature of restoring physiologic SORLA function, by applicable to treat patients at late stages of AD.

In some embodiments of the present disclosure, said construct, vector, cell and/or composition is administered to a patient at late stage of AD.

In some embodiments of the present disclosure, said construct, vector, cell and/or composition is administered to an individual when a relative of said individual is diagnosed with Alzheimer's disease.

In some embodiments of the present disclosure, said construct, vector, cell and/or composition is administered to an individual when it becomes known that a relative of said individual is suffering or has suffered from Alzheimer's disease.

It may, for example, become known that a relative of an individual is suffering from AD when said relative is diagnosed by a clinician. Alternatively, it may, for example become known that a relative of said individual is suffering or has suffered from Alzheimer's disease by obtaining information from other sources, such as family records or memories.

A relative of an individual may be understood as a family member of an individual.

A relative of an individual may be understood as a person sharing genetic material with said individual.

For example, a relative of an individual is a great-grandmother.

For example, a relative of an individual is a great-grandfather.

For example, a relative of an individual is a grandmother.

For example, a relative of an individual is a grandfather.

For example, a relative of an individual is a mother.

For example, a relative of an individual is a father.

For example, a relative of an individual is a sibling.

For example, a relative of an individual is a brother.

For example, a relative of an individual is a daughter.

For example, a relative of an individual is a son.

In some embodiments of the present disclosure, said construct, vector, cell and/or composition is administered to an individual once it is established that one or more family members of said individual suffer from Alzheimer's disease.

In some embodiments of the present disclosure said construct, vector, cell and/or composition is administered to an individual once it is established that one or more family members of said individual suffer from Alzheimer's disease.

The person skilled in the art will appreciate a potential genetic inherence of a risk to develop AD. The person skilled in the art will appreciate that administration of said construct, vector, cell and/or composition to an individual at risk for developing AD, e.g. having a risk when being a relative of a person diagnosed with AD, will offer, for example, the possibility of preventing AD prior to disease onset, or of reducing symptoms of AD.

In a further aspect, the present invention relates to a method of treating Alzheimer's Disease or a disease or disorder associated with Alzheimer's Disease, the method comprising administering to an individual in need thereof said construct, polypeptide P, vector, cell and/or composition.

In a further aspect, the present invention relates to the use of said construct, polypeptide P, vector, cell and/or composition for the manufacture of a medicament for the treatment, prevention and/or alleviation of Alzheimer's Disease, or a disease or disorder associated with Alzheimer's Disease.

In a further aspect, the present invention relates to the a method of increasing sAPPα in a cell, the method comprising introducing into a cell the polynucleotide construct of the invention, the vector of the invention, the polypeptide P of the invention and/or the composition of the invention, thus increasing sAPPα in said cell.

In a further aspect, the present invention relates to the a method of decreasing Aβ38, Aβ40, and/or Aβ42 in a cell, the method comprising introducing into a cell the polynucleotide construct of the invention, the vector of the invention, the polypeptide P of the invention and/or the composition of the invention, thus decreasing Aβ38, Aβ40, and/or Aβ42 in said cell.

The person skilled in the art will be able to select the available option for introducing the polynucleotide construct of the invention, the vector of the invention, the polypeptide P of the invention and/or the composition of the invention into a cell. For example, viral transfection may be used.

In some embodiments of the invention, said cell is a neuronal cell.

In some embodiments of the invention, said methods are in-vivo methods.

In some embodiments of the invention, said methods are in-vitro methods.

### EXAMPLES

### Example 1: Generation of the SORLA Mini-Receptor

### Aim

This example illustrates the generation of the SORLA-3Fn mini-receptor.

### Material and Methods

The mini-receptor construct was generated by a single PCR reaction (Herculase II Fusion) followed by restriction enzyme digestion and subsequent ligation. The primer pair (Fwd: 5′-CAGGATCCGACTACAAGGACCACGACGGCGACTACAAGGACCACGACATCGACT ACAAGGACGACGACGACAAGGAGTTGACTGTGTACAAAGTACAG-3′ and rev: 5′-GTGAATTCTCAGGCTATCACCATGG-3′) was used to amplify a human SORL1 fragment downstream of Glu1552 to the stop codon. Thus, the final amplicon encompassed a 5′ BamHI and a 3′ EcoRI restriction site flanking sequences for the FLAG-tag (3×), the 3Fn cassette (of six Fibronectin type III (FNIII or FN3) repeats, the transmembrane region and the cytoplasmic tail. Lastly, the mini-receptor PCR product was cloned into a Psectag2B expression vector (Invitrogen) between the BamHI and EcoRI restriction sites to be in frame with the murine lg κ-chain V-J2-C signal peptide. The expression product of the amplicon (the SORLA mini-receptor) is illustrated in FIG. 1, in comparison to full-length (wild-type) SORLA. The vector is depicted in FIG. 2.

Next, N2a cells were transfected with the vector encoding the mini-receptor to verify correct expression of the construct. Lysates from the transfected N2a cells were then harvested and proteins were analyzed by WB using antibodies recognizing the SORLA ectodomain (extracellular domain; targeting the respective protein product of SORL1-WT or SORL1-3Fn) and β-actin (FIG. 4).

Additionally, the domain boundaries of the individual SORLA 3Fn modules were determined in-silico using several softwares (PsiPred, SSpro Scratch and Yaspin) to predict secondary structures. Subsequently, an alignment of the six 3Fn domains was generated based upon their highly conserved secondary structures and few conserved amino acids (FIG. 3 C). FIG. 3A visualizes the secondary structure of a 3Fn domain. FIG. 3 B shows a schematic structure with conserved amino acids.

Note that during the herein described example, the mini-receptor has been constructed comprising a FLAG-tag, for example to be able to in a convenient way to trace the mini-receptor during the initial establishment and proof-of-concept phase. The person skilled in the art will be able to use the methodology described herein to establish a mini-receptor without any tag (or with an alternative tag) for use in a setting where a tag would not be needed or wanted, e.g. when using the mini-receptor in a therapeutical setting, e.g. when being expressed via a viral vector in patient cells.

### DNA Sequence of Full Mini-Receptor Construct:

BamHI + 3X-FLAG-TAG + 6xFN3 + transmembrane domain + cytoplasmic tail and stop
codon + EcoRI
Underlined: restriction sites
Nucleotides marked with grey are the last nucleotides of each domain

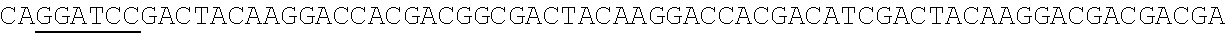

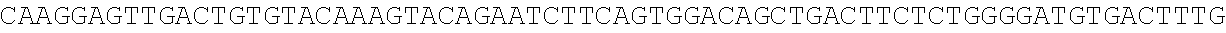

ACCTGGATGAGGCCCAAAAAAATGCCCTCTGCTTCTTGTGTATATAATGTCTACTACAGGGTGGTTGGAG
AGAGCATATGGAAGACTCTGGAGACCCACAGCAATAAGACAAACACTGTATTAAAAGTCTTGAAACCAGA
TACCACGTATCAGGTTAAAGTACAGGTTCAGTGTCTCAGCAAGGCACACAACACCAATGACTTTGTGACC

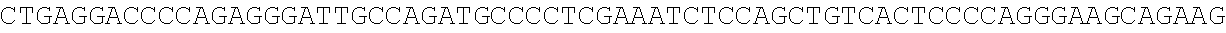

GTGTGATTGTAGGCCACTGGGCTCCTCCCATCCACACCCATGGCCTCATCCGTGAGTACATTGTAGAATA
CAGCAGGAGTGGTTCCAAGATGTGGGCCTCCCAGAGGGCTGCTAGTAACTTTACAGAAATCAAGAACTTA
TTGGTCAACACTCTATACACCGTCAGAGTGGCTGCGGTGACTAGTCGTGGAATAGGAAACTGGAGCGATT

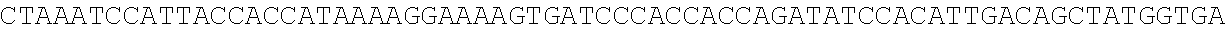

AAATTATCTAAGCTTCACCCTGACCATGGAGAGTGATATCAAGGTGAATGGCTATGTGGTGAACCTTTTC
TGGGCATTTGACACCCACAAGCAAGAGAGGAGAACTTTGAACTTCCGAGGAAGCATATTGTCACACAAAG
TTGGCAATCTGACAGCTCATACATCCTATGAGATTTCTGCCTGGGCCAAGACTGACTTGGGGGATAGCCC

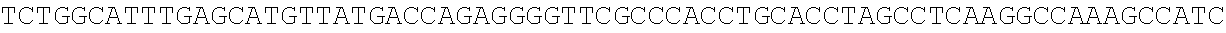

AACCAGACTGCAGTGGAATGTACCTGGACCGGCCCCCGGAATGTGGTTTATGGTATTTTCTATGCCACGT
CCTTTCTTGACCTCTATCGCAACCCGAAGAGCTTGACTACTTCACTCCACAACAAGACGGTCATTGTCAG
TAAGGATGAGCAGTATTTGTTTCTGGTCCGTGTAGTGGTACCCTACCAGGGGCCATCCTCTGACTACGTT

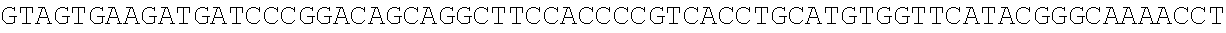

CCGTGGTCATCAAGTGGGAATCACCGTATGACTCTCCTGACCAGGACTTGTTGTATGCAATTGCAGTCAA
AGATCTCATAAGAAAGACTGACAGGAGCTACAAAGTAAAATCCCGTAACAGCACTGTGGAATACACCCTT
AACAAGTTGGAGCCTGGCGGGAAATACCACATCATTGTCCAACTGGGGAACATGAGCAAAGATTCCAGCA

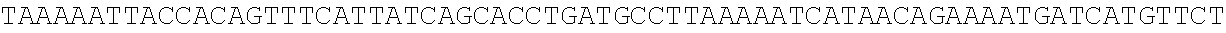

TCTGTTTTGGAAAAGCCTGGCTTTAAAGGAAAAGCATTTTAATGAAAGCAGGGGCTATGAGATACACATG
TTTGATAGTGCCATGAATATCACAGCTTACCTTGGGAATACTACTGACAATTTCTTTAAAATTTCCAACC
TGAAGATGGGTCATAATTACACGTTCACCGTCCAAGCAAGATGCCTTTTTGGCAACCAGATCTGTGGGGA

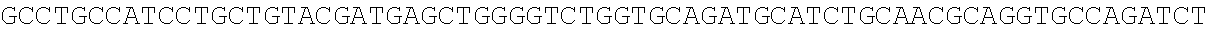

ACGGATGTTGCTGCTGTGGTGGTGCCCATCTTATTCCTGATACTGCTGAGCCTGGGGGTGGGGTTTGCCA

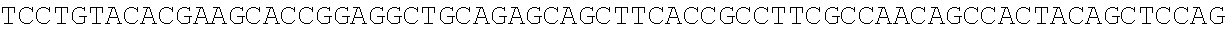

GCTGGGGTCCGCAATCTTCTCCTCTGGGGATGACCTGGGGGAAGATGATGAAGATGCCCCTATGATAACT

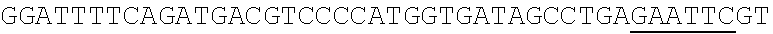

DNA sequence of the respective domains:
FnIII-1:
GAGTTGACTGTGTACAAAGTACAGAATCTTCAGTGGACAGCTGACTTCTCTGGGGATGTGACTTTGACCT
GGATGAGGCCCAAAAAAATGCCCTCTGCTTCTTGTGTATATAATGTCTACTACAGGGTGGTTGGAGAGAG
CATATGGAAGACTCTGGAGACCCACAGCAATAAGACAAACACTGTATTAAAAGTCTTGAAACCAGATACC
ACGTATCAGGTTAAAGTACAGGTTCAGTGTCTCAGCAAGGCACACAACACCAATGACTTTGTGACCCTGA
GGACCCCAGAGGGA
FnIII-2:
TTGCCAGATGCCCCTCGAAATCTCCAGCTGTCACTCCCCAGGGAAGCAGAAGGTGTGATTGTAGGCCACT
GGGCTCCTCCCATCCACACCCATGGCCTCATCCGTGAGTACATTGTAGAATACAGCAGGAGTGGTTCCAA
GATGTGGGCCTCCCAGAGGGCTGCTAGTAACTTTACAGAAATCAAGAACTTATTGGTCAACACTCTATAC
ACCGTCAGAGTGGCTGCGGTGACTAGTCGTGGAATAGGAAACTGGAGCGATTCTAAATCCATTACCACCA
TAAAAGGAAAA
FnIII-3:
GTGATCCCACCACCAGATATCCACATTGACAGCTATGGTGAAAATTATCTAAGCTTCACCCTGACCATGG
AGAGTGATATCAAGGTGAATGGCTATGTGGTGAACCTTTTCTGGGCATTTGACACCCACAAGCAAGAGAG
GAGAACTTTGAACTTCCGAGGAAGCATATTGTCACACAAAGTTGGCAATCTGACAGCTCATACATCCTAT
GAGATTTCTGCCTGGGCCAAGACTGACTTGGGGGATAGCCCTCTGGCATTTGAGCATGTTATGACCAGA
FnIII-4:
GGGGTTCGCCCACCTGCACCTAGCCTCAAGGCCAAAGCCATCAACCAGACTGCAGTGGAATGTACCTGGA
CCGGCCCCCGGAATGTGGTTTATGGTATTTTCTATGCCACGTCCTTTCTTGACCTCTATCGCAACCCGAA
GAGCTTGACTACTTCACTCCACAACAAGACGGTCATTGTCAGTAAGGATGAGCAGTATTTGTTTCTGGTC
CGTGTAGTGGTACCCTACCAGGGGCCATCCTCTGACTACGTTGTAGTGAAGATGATCCCGGACAGC
FnIII-5:
AGGCTTCCACCCCGTCACCTGCATGTGGTTCATACGGGCAAAACCTCCGTGGTCATCAAGTGGGAATCAC
CGTATGACTCTCCTGACCAGGACTTGTTGTATGCAATTGCAGTCAAAGATCTCATAAGAAAGACTGACAG
GAGCTACAAAGTAAAATCCCGTAACAGCACTGTGGAATACACCCTTAACAAGTTGGAGCCTGGCGGGAAA
TACCACATCATTGTCCAACTGGGGAACATGAGCAAAGATTCCAGCATAAAAATTACCACAGTTTCATTA
FnIII-6:
TCAGCACCTGATGCCTTAAAAATCATAACAGAAAATGATCATGTTCTTCTGTTTTGGAAAAGCCTGGCTT
TAAAGGAAAAGCATTTTAATGAAAGCAGGGGCTATGAGATACACATGTTTGATAGTGCCATGAATATCAC
AGCTTACCTTGGGAATACTACTGACAATTTCTTTAAAATTTCCAACCTGAAGATGGGTCATAATTACACG
TTCACCGTCCAAGCAAGATGCCTTTTTGGCAACCAGATCTGTGGGGAGCCTGCCATCCTGCTGTACGATG
AGCTGGGGTCT
Transmembrane domain:
GGTGCAGATGCATCTGCAACGCAGGCTGCCAGATCTACGGATGTTGCTGCTGTGGTGGTGCCCATCTTAT
TCCTGATACTGCTGAGCCTGGGGGTGGGGTTTGCCATCCTGTACACG
Cytoplasmic tail (stop codon underlined):
AAGCACCGGAGGCTGCAGAGCAGCTTCACCGCCTTCGCCAACAGCCACTACAGCTCCAGGCTGGGGTCCG
CAATCTTCTCCTCTGGGGATGACCTGGGGGAAGATGATGAAGATGCCCCTATGATAACTGGATTTTCAGA
TGACGTCCCCATGGTGATAGCCTGA
Amino acid sequence of the full mini-receptor construct (including FLAG-tag)

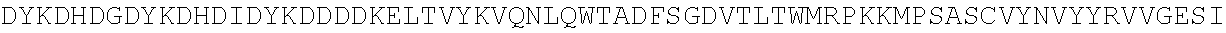

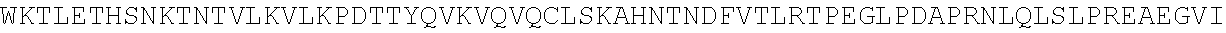

VGHWAPPIHTHGLIREYIVEYSRSGSKMWASQRAASNFTEI KNLLVNTLYTVRVAAVTSRGIGNWSDSKS

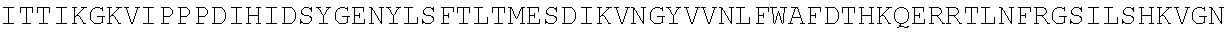

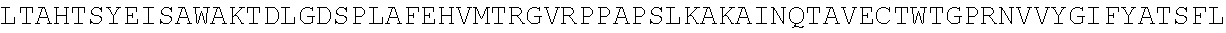

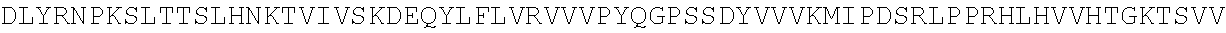

IKWESPYDSPDQDLLYAIAVKDLIRKTDRSYKVKSRNSTVEYTLNKLEPGGKYHIIVQLGNMSKDSSIKI

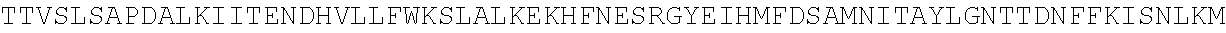

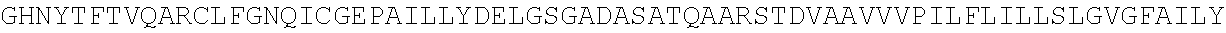

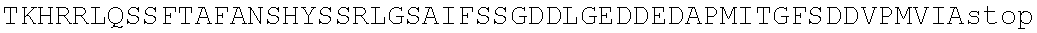

Amino acids highlighted in grey are the last amino acids of each domain.
FnIII mini-receptor: 685 amino acids (FLAG-tag, FNIII cassette, transmembrane
domain, cytoplasmic domain)
FLAG:
DYKDHDGDYKDHDIDYKDDDDK
FnIII-1: 1552-1649
ELTVYKVQNLQWTADFSGDVTLTWMRPKKMPSASCVYNVYYRVVGESIWKTLETHSNKTNTVLKVLKPDT
TYQVKVQVQCLSKAHNTNDFVTLRTPEG
FnIII-2: 1650-1746
LPDAPRNLQLSLPREAEGVIVGHWAPPIHTHGLIREYIVEYSRSGSKMWASQRAASNFTEIKNLLVNTLY
TVRVAAVTSRGIGNWSDSKSITTIKGK
FnIII-3: 1747-1839
VIPPPDIHIDSYGENYLSFTLTMESDIKVNGYVVNLFWAFDTHKQERRTLNFRGSILSHKVGNLTAHTSY
EISAWAKTDLGDSPLAFEHVMTR
FnIII-4: 1840-1931
GVRPPAPSLKAKAINQTAVECTWTGPRNVVYGIFYATSFLDLYRNPKSLTTSLHNKTVIVSKDEQYLFLV
RVVVPYQGPSSDYVVVKMIPDS
FnIII-5: 1932-2024
RLPPRHLHVVHTGKTSVVIKWESPYDSPDQDLLYAIAVKDLIRKTDRSYKVKSRNSTVEYTLNKLEPGGK
YHIIVQLGNMSKDSSIKITTVSL
FnIII-6: 2025-2121
SAPDALKIITENDHVLLFWKSLALKEKHFNESRGYEIHMFDSAMNITAYLGNTTDNFFKISNLKMGHNYT
FTVQARCLFGNQICGEPAILLYDELGS
Transmembrane domain: 2122-2160
GADASATQAARSTDVAAVVVPILFLILLSLGVGFAILYT
Cytoplasmic tail: 2161-2214

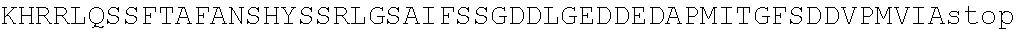

Numbers are amino acid positions from the human SORLA full-length protein.

### Results

This cloning experiment demonstrates the successful generation of a SORL1 mini-gene and expression of a SORLA mini-receptor consisting of a FLAG-tag (3×), six 3Fn modules, a transmembrane domain and the cytoplasmic tail. A schematic overview of the produced mini-receptor, in comparison with SORLA-wt (transcribed from SORL1-WT), is depicted in FIG. 1. The mini-receptor (SORL1-3Fn) migrated on an SDS-PAGE gel with an actual molecular size of around 100 kDa which is larger than the theoretical mass of 74 kDa (FIG. 4). The size difference is likely attributed to glycosylations since the 3Fn domains contain several glycosylation sites.

### Conclusion

In summary, we demonstrated for the first time the successful production and expression of a SORLA-3Fn mini-receptor encompassing the 3Fn-cassette, the transmembrane region and the cytoplasmic tail. This mini-receptor is used in the following examples to functionally characterize the invention.

### Example 2: The SORLA Mini-Receptor Specifically Decreases the Level of the Amyloidogenic Products

### Aim

This example aimed to investigate how the SORLA mini-receptor affects key proteins and pathways involved in Alzheimer's disease. We have previously shown how the full-length SORLA protein decreases APP trafficking in the secretory pathway and increases APP recycling out of the endosome resulting in a decrease of both sAPPα and sAPPb/Ab-peptide products (Andersen et al. 2005). However, as the mini-receptor does not harbor the CR-domains required for the direct binding of the APP protein (Mehmedbasic et al. 2015), nor the VPS10p-domain suggested to bind the amyloid-β (Aβ) peptide (Kitago et al. 2015), it is not known whether the remaining part of SORLA that constitute the mini-receptor also has an impact on APP biology.

### Material and Methods

To address this, we transfected Murine neuroblastoma-2a cells (N2a) cells with either a construct for human APP-myc for expression of the human APP protein (together with a pcDNA plasmid without insert) or co-transfected cells with APP-myc together with plasmids encoding SORLA-wt or SORLA-3Fn (SORLA mini-receptor, as described in Example 1, or also referred to as FLAG-mini in Example 8) and analyzed APP proteolysis using a western blot approach and quantification of specific APP metabolites by mesoscale discovery (MSD) assays specific for human-sequence APP fragments (FIG. 4, FIG. 6 and FIG. 8). The myc-tag allows for easy detection of APP-myc by an anti-myc antibody. 48 hs post-transfection, medium was changed to serum-free medium and left on the cells for another 48 hours before collecting the conditioned medium and harvesting cells. Proteins in cell lysates and medium were separated using SDS-PAGE and blotted onto nitrocellulose membranes. Wb analysis was performed using antibodies for detection of APP-myc, SORLA and β-actin (as loading control) in cell lysates or sAPPα in the medium samples (FIG. 4). The experiment was repeated at least 5 times with duplicate cell transfections. Bands of similar intensity corresponding to 250 kDa and 100 kDa representing full-length SORLA and mini-receptor SORLA were clearly visible using a polyclonal antibody against the entire SORLA extracellular domain (sol-SORLA), showing similar transfection efficiency of the two SORLA constructs.

### Results

As previously shown (Andersen et al. 2005), SORLA-wt (transcribed from SORL1-WT) inhibits cleavage of APP into sAPPα (lanes 5 and 6) leading to a strong reduction of the signal for sAPPα in conditioned medium from SORLA-wt transfected cells (FIG. 4 B).

However, cells transfected with SORLA-3Fn (transcribed from SORL1-3Fn) did not show any obvious reduction of sAPPα levels in conditioned medium (lanes 7 and 8) based on inspection of the Western Blots (Wb) (FIG. 4).

Consequently, there is a substantial difference in how SORLA-wt and the mini-receptor effect the levels of sAPPα. The mini-receptor is superior to SORLA-wt in leading to cleaved APP—leading to increased sAPPα, and in not decreasing sAPPα. This is beneficial for the AD patient.

These results were further confirmed by quantification of band intensities from all five Wb membranes, showing that no decrease of sAPPα could be detected in cells transfected with mini-receptor. The level of sAPPα was even higher than for control cells (FIG. 5).

This finding was validated by measuring sAPPα with the more quantitative MSD analysis (FIG. 6A). The assay also allowed quantification of sAPPβ from the samples. While sAPPα was increased in the presence of the mini-receptor, the level of sAPPβ was lower than observed for control cells (FIG. 6 B), suggesting a unique function of the mini-receptor in selectively lowering the amyloidogenic processing of APP. In other words, amyloidogenic processing of APP encompasses the production of sAPPβ while non-amyloidogenic processing of APP encompasses the production of sAPPα. The herein presented data shows that the minireceptor supports non-amyloidogenic processing.

Accordingly, besides the size of the mini-receptor construct enabling viral delivery (i.e. gene therapy), the specific decrease of the amyloidogenic processing is an advantage as the non-amyloidogenic sAPPα fragment is considered to have neurotrophic/beneficial effects.

We therefore next determined the level of Aβ40 in the medium from the transfected N2a cells using a standard ELISA protocol (five samples selected from three independent experiments). As shown previously (Andersen et al. 2005), the level of Aβ40 is strongly reduced in the presence of SORLA-wt (FIG. 7). However, the level of Aβ40 was also reduced in the presence of SORLA-3Fn compared to control cells, in line with the observation that sAPPβ was lowered in medium from SORLA-3Fn transfected cells (see FIG. 6 B).

This finding was substantiated by MSD quantification of the three Amyloid β-peptide species Aβ38, Aβ40, and Aβ42 where we found a significant decrease of all three Aβ forms in the presence of SORLA-3Fn compared to control cells, to a similar low level as seen for cells transfected with SORLA-wt (FIG. 8A-C). In general, Aβ38, Aβ40, and Aβ42 are regarded as reporters of unsufficient endosomal state/low endosomal activity. Thus, we assess that a reduction of these markers suggests a restoration of endosomal activity.

In summary, in line with previous findings, the presence of SORLA-wt decreased both non-amyloidogenic and amyloidogenic processing, as evidenced by significantly reduced levels of sAPPα, sAPPb, and Ab-peptides of 38, 40, and 42 amino acids in length (FIG. 8A-C). Rather to our surprise, we also observed a significant decrease on APP amyloidogenic processing in the presence of FLAG-mini, with significantly decreased production of sAPPb and all three Ab peptides (Ab38/Ab40/Ab42). However, whereas the full-length SORLA-wt also decreased sAPPα production, FLAG-mini significantly increased this non-amyloidogenic fragment (FIG. 6A).

### Conclusion

Whereas SORLA-wt decreases both the amyloidogenic and the non-amyloidogenic processing of APP, the mini-receptor SORLA-3Fn has no effect on the non-amyloidogenic processing (as sAPPα is not altered). However, the mini-receptor can specifically decrease the level of the amyloidogenic products (sAPPβ and Aβ). This profile is similar to the situation observed for cells treated with retromer enhancing drugs (Mecozzi et al. 2014) since retromer function is related to trafficking cargo (i.e. APP) out of the endosome which is the main cellular site for the amyloidogenic processing (Haass 2012). As the mini-receptor shares the same cytoplasmic domain as SORLA-wt, the mini-receptor is likely to also engage in retromer-dependent endosomal recycling similar to SORLA-wt (Fjorback et al. 2012). The SORLA-dependent reduction in non-amyloidogenic processing of APP into sAPPα relies on SORLA-wt (containing the CR-cluster) activity in the secretory pathway (Mehmedbasic et al. 2015). Taken together this provides a model that suggest independent roles of SORLA in these two processing pathways. This is the optimal profile for a drug as sAPPα is speculated to have a neurotrophic/beneficial function, and thus pharmacological interventions would optimally be designed not to affect the generation of this APP fragment.

It is a surprise and unexpected benefit that the minireceptor SORLA-3Fn is able to decrease the amyloidogenic processing of APP (via decrease of sAPPβ) as a previous study showed how SORLA deleted of its 11 CR-domains, but with an intact 3Fn-region, did not interact with APP nor its processing (Mehmedbasic et al. 2015). The herein presented data supports a promising therapeutic effect of delivering the minireceptor to the brain of AD patients.

We speculate that the mini-receptor forms a complex with APP and BACE1 as previously described for SORLA-wt (Spoelgen et al. 2006), where we also found that the cytoplasmic domain of SORLA was able to decrease formation of this ternary complex.

### Example 3: Introduction of Mutations in the FANSHY Motif of the Mini-Receptor Construct to Generate 2nd Generation Mini-Receptors with Improved Retromer-Dependent Trafficking Properties

### Aim:

We will provide proof-of-concept that some mutations in the FANSHY sequence can increase complex-formation with retromer, and that such mutated constructs have improved ability to undergo endosomal trafficking and to further decrease APP amyloidogenic processing.

### Material and Methods

We will use a panel of peptides comprising the FANSHY motif. The peptides will either carry the wildtype FANSHY sequence, or substitution(s) of single amino acid(s) that, based on in silico analysis, are predicted to increase affinity for retromer.

Such peptides will be tested for interaction with retromer using isothermal titration calorimetry.

Mutations that lead to increased retromer binding of peptides will be introduced in constructs for SORLA-3Fn for subsequent testing of effects on APP processing in cultured (N2a) cells and/or primary murine neuronal cultures.

### Results

We expect to identify a variant or several variants of the mini-receptor that bind(s) stronger to retromer, and that decreases APP amyloidogenic processing even more than the mini-receptor without the above described modifications (i.e. substitutions).

### Conclusion

2^{nd} generation mini-receptor(s) will have improved translational value as they increase endosomal recycling even more and show an even stronger reduction in Aβ levels as compared to the mini-receptor without the above described modifications (i.e. substitutions).

### Example 4: A Method for Viral Delivery of SORLA-3Fn to Cultures of Primary Neurons from Mice and Human iPSC-Derived Neurons

### Aim:

Providing evidence that viral delivery of the mini-receptor reduces amyloidogenic processing in neuronal cultures as it does in cultured cell lines.

### Material and Methods

First we will prepare a AAV9-based viral vector with the SORLA-3Fn insert (with or without any tag). Next, we will isolate primary cortical (or hippocampal) neurons from wild-type mice, and compare APP processing in infected versus non-infected neurons using Western blotting and mesoscale discovery assays for the endogenous mouse APP fragments.

### Results

We expect to find decreased Aβ secretion of infected neurons compared to non-infected neurons, but similar levels of sAPPα. The successful delivery of SORLA-3Fn to primary neurons will be shown by Western blot of cell lysates using an antibody for the SORLA extracellular domain.

### Conclusion

We expect to show that the mini-receptor can be used to decrease Aβ levels in primary neurons.

### Example 5: Rescue of Endosomal Enlargement

### Aim:

To demonstrate that the SORLA mini-receptor can reduce the enlarged endosomes observed in SORL1 deficient neuronal cultures similar to previous findings relating to retromer-enhancing compounds (Jessica Young, unpublished).

### Material and Methods

We will investigate, using iPSC-derived neurons with SORL1 knockout (as published by Knupp et al, 2020), if mini-receptor treatment will rescue endosome enlargement. If so, we would next apply similar “rescue-experiments” to iPSC-derived neurons carrying disease alleles for APP and/or PSEN1 that also show increased (i.e. pathogenic) endosome size (Kwart et al. 2019).

The iPSC line with targeted deletion of SORL1 has been instablished (Knupp et al. 2020) (8). We will determine endosome size using Rab5 immunocytochemistry in SORL1-deficient neurons that has been infected with SORLA-3Fn mini-receptor, and compare to non-infected neurons. Subsequently, iPSC-derived neurons with other AD-related mutations will be infected with the SORLA-3Fn mini-receptor.

### Results

We expect to find that SORLA-3Fn reduces the size of endosomes of SORL1-deficient iPSC-derived neurons to a similar size of wildtype iPSC-derived neurons (i.e. rescue). In iPSC-derived neurons with causal variants in the genes of APP or PSEN1, infection with SORLA-3Fn similarly restores normal endosome size.

### Conclusion

We expect to find that delivery of the gene for the SORLA mini-receptor can reduce pathologically enlarged endosomes in neurons that are genetically similar to causal variants, i.e. deficient of SORL1 or with mutations in APP and PSEN1.

### Example 6: Stabilization of the Retromer Complex by the Mini-Receptor

### Aim:

Further development of the mini-receptor.

### BACKGROUND

Structures elucidated by cryoEM of the retromer complex assembled at endosomal tubule membranes have identified a coat of retromer at the cytosolic site, where two core retromer units form an arch that dimerizes by the VPS29 subunit at the top, and where two VPS26 subunits are also in close contact at the bottom (FIG. 9A). This forms the basic unit for the coat structure. Previous studies have identified that the cytoplasmic tail of SORLA interacts with VPS26 (Fjorback 2012). We have now identified that the 3Fn-domains of SORLA (and of mini-receptor) is able to dimerize (FIG. 9 B). It is therefore reasonable to assume that the mini-receptor, when expressed in cells, forms dimers at the endosomal membrane where two cytoplasmic tails interact with two neighboring VPS26 subunits of the retromer complex. As the mini-receptor induces dimerization, the presence of the mini-receptor will lead to stabilization of the retromer-coat and endosomal tubular activity. Consequently, the mini-receptor will stabilize the retromer complex. Increased retromer stability and thus activity has been demonstrated to decrease the amyloidogenic cleavage of APP, with a concomitant increase in the generation of the non-amyloidogenic product soluble APPα (sAPPα) (Mecozzi et al. 2014).

### Material and Methods

Methods to increase dimerization of the mini-receptor will be tested for the ability to increase its protective activity against AD. In one setting, aimed for gene therapy with the mini-receptor, a mini-receptor version with engineered mutations that increase the strength of the dimer will established. This version will be superior in activity compared to the non-engineered mini-receptor. Such mutations may be identified based on the structure of the mini-receptor dimer complex.

Compounds (small molecule chaperones or antibodies) that enter the endosomal lumen by endocytic uptake, and that can increase the SORLA 3Fn-dimer interaction, may further increase the strength of the mini-receptor dimer. This will be tested.

### Results

We will further optimize the mini-receptor.

### Conclusion

Different versions of the mini-receptor will be established, with different fine-tuned properties.

### Example 7: FANSHY-Motif Dependent Intracellular Trafficking of the SORL1 Mini-Receptor

### Aim:

To demonstrate that the FANSHY-motif in the SORLA cytoplasmic tail, previously shown to be involved in binding to Retromer, is also important for the intracellular trafficking of the SORLA minireceptor.

### Material and Methods

A construct for FLAG-mini-WT (as referred to as SORLA-3Fn mini-receptor in Examples 1 and 2, comprising an N-terminal FLAG tag, WT=wildtype indicating the non-mutated variant of the minireceptor in Example 7) was used as template for site-directed mutagenesis to generate a mutant construct (FLAG-mini-FANSHY) with the six amino acid FANSHY-motif being substituted by alanine residues, using standard methods and primers.

N2a cells were transfected with the two constructs using FuGene and, following standard protocols, lysates and medium were collected 48 hours after transfection and analysed by SDS-PAGE and Western blot (Wb) experiments using a polyclonal antibody that recognizes the extracellular fragment of full-length SORLA, and which is therefore also able to bind the extracellular part of the mini-receptors (FIGS. 10A and 10B). Quantifications of signal from 5 independent experiments were plotted using Prism 6.0 software (FIG. 10C).

FACS analysis of transfected cells were conducted using standard protocols and the same primary polyclonal SORLA antibody as for Western blot analysis was used for cell staining, as well as a fluorescently labelled secondary anti-rabbit antibody coupled to fluorochrome 488 (FIG. 10D and FIG. 10E).

Immunocytochemical detection of cell surface expression of transfected cells was also performed using the polyclonal anti-SORLA antibody. Detergent has been left out of all solutions for incubation and washing to avoid penetration of antibodies across the cell membrane. Therefore, the antibody is expected to only bind receptor molecules located at the cell surface (FIG. 10F).

### Results

We generated a mutant mini-receptor, FLAG-mini-FANSHY, that is unable to bind Retromer as the FANSHY-motif has been replaced by alanine residues as previously described (Fjorback, 2012). We used lysates and conditioned medium from cells transfected with either FLAG-mini-WT (mini-receptor without any mutations in the tail region, equivalent to SORLA-3Fn mini-receptor referred to in Examples 1 and 2) or FLAG-mini-FANSHY (mini-receptor with mutations in the tail region) to determine how shedding from the cell surface was affected by lack of Retromer binding. Whereas we did not observe any difference on the cellular expression between the two mini-receptor variants (FIG. 10A), we found a significant increase from the secreted mutated mini-receptor variant, compared to the non-mutated variant (FIG. 10B), which is likely to be the result of increased shedding at the cellular surface by proteolytic cleavage by TACE in a process similar but enhanced as observed for the full-length SORLA protein. This suggest that deletion of the Retromer binding site in the mutated variant reduced the ability of the mini-receptor to locate intracellularly, i.e. in endosome structures, and consequently increases the cell surface localization. This further supports the hypothesis that the mini-receptor stabilizes endosomes.

In order to obtain support for a change in cellular distribution, we applied Fluorescence-activated cell sorting (FACS) analysis to determine the expression at the cell surface of FLAG-mini-WT and FLAG-mini-FANSHY. Using this method we analyzed the cell surface expression for two samples of transfected cells and found more cells with the mutant construct at the cell surface (WT: 321/347 vs FANSHY: 592/509) as well as much stronger signal (WT: 4.63E6/4.73E6 vs FANSHY: 5.29E6/5.29E6) for the mutated mini-receptor (FIG. 10D). In general terms, about 50% more cells have the mini-receptor on the cell surface, i.e. when comparing cells transfected with the mutant variant FLAG-mini-FANSHY compared to cells transfected with the non-mutant variant FLAG-mini-WT. Moreover, the expression levels of the mini-receptor was also elevated by around 20% for the mini-receptor deleted of its Retromer bindingsite (i.e. FLAG-mini-FANSHY).

To visualize the stronger expression of FLAG-mini-FANSHY at the cell surface in comparison to FLAG-mini-WT, we also performed immunocytochemical analysis of transfected cells and determined cell surface localization by acquiring confocal microscopy pictures of cells stained with primary antibody for SORLA in the absence of a detergent allowing to identify only mini-receptor at the cell surface. Representative pictures show how the cell surface signal for the mutated variant FLAG-mini-FANSHY is stronger than for the non-mutated variant FLAG-mini-WT (FIG. 10E), which is in agreement with the result from FACS.

### Conclusion

We have provided evidence that deletion of the Retromer binding site of the FLAG-mini-receptor decreases the intracellular localization of the construct, and increases its expression at the cell surface. This further confirms the ability of a mini-receptor construct (e.g. as described in Examples 1 and 2) to interact with retromer and to perform important functions needed to ensure physiological endosomal processing. As such the mini-receptor is expected to be functional and is expected to be beneficial in a therapeutic setting, e.g. via viral transfer to Alzheimer's Disease patients.

### Example 8: 3Fn Mediated Dimerization of SorLA or the SorLA Mini-Receptor is Essential for its Interaction with Retromer Regulating Endosomal Trafficking

### Aim:

To further investigate SorLA as well as SorLA mini-receptor binding and dimerization capabilities.

### Material and Methods

### Cloning

The domain boundaries of the SORL1 3Fn cassette was determined in-silico using several online softwares (PsiPred, SSpro, Scratch and Yaspin) to predict secondary structures in the amino acid sequence. Subsequently, an alignment of the six SORL1 3Fn domains was generated based on their highly conserved pattern of secondary structures and few conserved amino acids.

The FLAG 3Fn minireceptor was generated by a single-step PCR reaction (Herculase=Fusion) using the primer pair (fwd: 5′-CAGGATCCGACTACAAGGACCACGACGGCGACTACAAGGACCACGACATCGACT ACAAGGACGACGACGACAAGGAGTTGACTGTGTACAAAGTACAG-3′ and rev: 5′-GTGAATTCTCAGGCTATCACCATGG-3′) amplifying a human SORL1 fragment downstream of Glu1552 (predicted beginning of 3Fn cassette) to the stop codon. Additionally, the forward primer adds a sequence encoding a 3×FLAG tag to the 5′ end of the amplicon. The PCR product was cloned into a Psectag2B expression vector (Invitrogen) using BamHI and EcoRI restriction enzymes utilizing the murine lg κ-chain V-J2-C signal peptide to drive expression.

The myc 3Fn minireceptor was produced similarly by using the primer pair (fwd: 5′-CAGGATCCGAGTTGACTGTGTACAAAGTAC-3′ and rev: 5′-GTGAATTCTCACAGATCCTCTTCTGAGATGAGTTTTTGTTCGGCTATCACCATGGG-3′) adding a myc-tag to the C-terminal.

### Site-Directed Mutagenesis

The soluble 3Fn minireceptor with a FLAG-tag was generated by site-directed mutagenesis introducing a stop codon in the predicted C-terminal of the 3Fn cassette. Primers were designed following instructions from the manufacturer (fwd: 5′-CCTGCTGTACGATGAGTGAGGGTCTGGTGCAGATGC-3′, rev: 5′-GCATCTGCACCAGACCCTCACTCATCGTACAGCAGG-3′). Site-directed mutagenesis was performed on human cDNA (3Fn minireceptor) within the psectag2B vector using the Quickchange XL site-directed mutagenesis kit (Agilent).

### Cell Culture

HEK293, N2a and Hela cells were cultured in Dulbecco's Modified Eagles Medium (DMEM, Lonza) supplemented with 10% fetal bovine serum (FBS) and 5% penicillin/streptomycin (P/S) in a humidified 5% CO2 incubator at 37° C.

CHO cells were grown in Hyclone (Sigma) medium supplemented with 10% FBS and 5% P/S in a humidified 5% CO2 incubator at 37° C.

Transient transfections of cells with different SORL1 and minireceptor constructs were performed using Fugene 6 Transfection Reagent kit (Promega) and either 1 μg or 3 μg of DNA templates.

### APP Metabolism

Approximately 5×10⁵ N2a cells were seeded per well in a 6-well plate and grown for 24 hrs. Cells were then transiently co-transfected with plasmids encoding APP-myc together with either SORL1-WT, 3Fn minireceptor or an empty pcDNA3.1 vector. The medium was changed to serum-free medium (SFM) after 48 hrs and incubated for another 48 hrs before harvesting cell lysates and conditioned medium.

### Immunoblotting

Cell supernatant was collected, and cell pellets were lysed on ice for 1 h in lysis buffer (1M Tris HCl pH 8.1, 0.5M EDTA, 1% Triton-X-100, 1% NP40) supplemented with Complete™ protease inhibitor (Roche). Total protein content in the lysates was measured using the BCA assay kit (Sigma).

Lysate and media samples were mixed with SDS and 5% β-mercaptoethanol (unless otherwise noted) followed by heating at 95° C. for 5 min. Proteins were separated by SDS-PAGE gel electrophoresis using precast 4-12% Bis-Tris gradient gels (Invitrogen) and transferred onto a nitrocellulose membrane (Amersham). The membranes were blocked for 1 h at RT in blocking buffer (Tris-Base 0.25M, NaCl 2.5M, skimmed milk 2%, tween-20 2%) followed by incubation in primary antibodies O.N. at 4° C. Membranes were incubated in HRP-conjugated secondary antibodies (1:1500) for 1 h at RT and finally the proteins were detected using SuperSignal West Femto Maximum Sensitivity Substrate (Thermofisher) on a Las-4000 Image Reader (Fujifilm).

Antibodies: anti-FLAG (M2, Sigma), anti-sAPPα (WO2) (MABN10, Merck), anti-myc (R951-25, Invitrogen), anti-LR11 (BD Transduction Laboratories), anti-SORLA generated to recognize the entire extracellular part of the SORLA proteins (5387, in house, Aarhus University), anti-VPS26b, anti-APP and anti-β-actin (A5441, Sigma).

### SAPPα/sAPPβ Immunoassay (Mesoscale)

The level of shed sAPPα and sAPPβ in supernatant from transfected N2a cells was measured using the sAPPα/sAPPβ duplex assay kit (Mesoscale Discovery).

A serial dilution of the sAPPα and sAPPβ calibrators was prepared following instructions from the manufacturer. The precoated 4-spot plate was incubated at RT in 3% blocker A solution for 1 h with shaking followed by 3 times washing in Tris wash buffer. Calibrators and undiluted cell supernatants (25 μl) were added to the plate and incubated for 1 h at RT with shaking and subsequently the plate was washed 3 times. Finally, the wells were incubated in 1× Read Buffer T for 10 min at RT followed by detection of electrochemilumiscent signals from the samples using a SECTOR® Imager (Mesoscale Discovery). Concentrations of sAPPα and sAPPβ were calculated on Microsoft excel by fitting to a standard curve generated from the calibrators.

### Aβ Peptide Panel 1 Immunoassay (Mesoscale)

Secreted Aβ38, Aβ40 and Aβ42 in cell supernatants from transfected N2a cells were quantified by the Aβ panel 1 V-plex assay kit (Mesoscale Discovery).

Aβ38, Aβ40 and Aβ42 calibrators were mixed and diluted in a 4-fold serial dilution as instructed from the manufacturer. Precoated wells of the multi-spot plate were blocked at RT in Diluent 35 for 1 h at RT and washed 3 times in 1×PBS+0.05% Tween-20. Detection antibody and calibrators/undiluted cell supernatants were added simultaneously to the plate and incubated for 2 hrs at RT with shaking. Lastly, the wells were washed 3 times and 2× Read Buffer T was added for immediate analysis of electrochemiluminescent signals on a SECTOR® Imager. Concentrations of Aβ38, Aβ40 and Aβ42 were calculated by Microsoft excel by fitting the samples to a standard curve.

### Co-Immunoprecipitation

HEK293 cells were transiently transfected followed by 48 hrs of incubation before harvesting cell lysates. Gammabind G sepharose beads/dynabeads were coated with 2.5 μg of anti-FLAG antibody (F1804, Sigma) for 2 hrs with end-over-end rotation at RT. Beads were washed 3 times and incubated in 70 μg of total protein from transfected HEK293 cells O.N. at 4° C. with end-over-end rotation. Next, beads were washed 5 times and bound proteins were eluted from the beads using SDS sample buffer supplemented with 5% β-mercaptoethanol. All washing steps were performed in PBS+0, 05% tween-20.

### Immunocytochemistry and Confocal Microscopy

Approximately 5×10⁴ HEK293 cells were seeded on glass coverslips coated with poly-L-lysine 0.1% (Sigma) followed by 24 hrs of incubation before transfection. Cells were transfected with SORL1-FLAG and SORL1-GFP using Fugene (Promega) transfection reagent. After another 24 hrs cells were fixed in 4% PFA for 10 min at RT with subsequent washing in PBS. Coverslips were washed in PBS with 0.1% triton-x-100 and blocked in PBS supplemented with 10% FBS for 30 min at RT. After blocking, the cells were incubated O.N. at 4° C. with primary antibodies against the FLAG-tag (F1804 Sigma, 1:1000) and the GFP-tag (ab6556 Abcam, 1:2000). Next, cells were washed in PBS with 0.1% triton-x-100 and incubated in Alexa fluor secondary antibodies (Invitrogen, 1:500) for 1 hr at RT. Cells were then washed in PBS and nuclei were counterstained with Hoechst (Abcam, 1:50000) for 10 min at RT. Lastly, the coverslips were mounted on glass slides with DAKO fluorescence mounting medium (Agilent), and after drying stained cells were imaged using a Zeiss LSM780 confocal microscope.

### Bimolecular Fluorescence Complementation (BiFC)

Approximately 5×10⁴ HEK293 cells were seeded on glass coverslips coated with poly-L-lysine 0.1% and grown for 24 hrs before transfection using Fugene (Promega). Cells were transfected with 500 ng of either SORL1-V1, SORL1-V2, SORL1-GFP or both SORL1-V1 and SORL1-V2. Transfected cells were incubated for another 24 hrs and fixed with 4% PFA for 10 min followed by washing in PBS. Counter-staining with SORLA antibody (sol-SORLA, 5367) diluted 1:100 and DAPI was performed as described for ICC above. The coverslips were mounted on glass slides using DAKO mounting medium (Agilent) and cells were visualized on a Zeiss LSM780 confocal microscope.

### In Situ Proximity Ligation Assay (PLA)

HEK293 cells were seeded on 0.1% poly-L-lysine coated glass coverslips and incubated for 24 hours before transfection using Fugene (Promega). The cells were co-transfected with 500 ng plasmids encoding either SORL1-FLAG or SORL1-GFP and incubated for 24 hours. The transfected cells were fixed in 4% PFA for 10 min and washed in PBS followed by incubation in primary antibodies. Cells were incubated overnight at 4° C. in either an anti-FLAG antibody (F1804 M2, Sigma) or an anti-GFP antibody (ab5665 rabbit, Abcam) or both diluted 1:1000 and 1:2000 respectively. The cells were then washed in PBS with 0.1% triton-x-100 and incubated in PLA probes (Sigma) for 1 hr at 37° C. Next, cells were washed in Wash buffer A and incubated in ligase (Sigma) for 30 min at 37° C. The coverslips were then washed in buffer A followed by incubation in polymerase (Sigma) for 100 min at 37° C. Subsequently, the cells were first washed in wash buffer B followed by washing in 0.01×wash buffer B for 1 min. Finally, coverslips were mounted on glass slides with Duolink® In Situ Mounting Medium with DAPI (Sigma) and imaged by confocal microscopy (LSM780, Zeiss).

### GFP-Trap Immunoprecipitation

SORL1 dimers formed by the interaction between C-terminal venus fragments (and SORL1-GFP as control) were immunoprecipitated using GFP-Trap Magnetic Particles (M-270, Chromotek) following the manufacturer's instructions. 500 ng of total lysate from transfected HEK293/Hela cells was incubated with 25 μl of magnetic bead slurry for 1 hr at 4° C. with end-over-end rotation. Subsequently, beads were washed 5 times in wash buffer (10 mM Tris/HCl PH 7.5, 150 mM NaCl, 0.05% Nonidet-P40, 0.5 mM EDTA) and eluated using SDS sample buffer.

### Results

### SORLA Forms a Dimer by 3Fn-Domains In Vitro

To further delineate the dimer capacity of SORLA, we generated two SORLA minireceptor constructs comprising the six 3Fn-domains, the transmembrane region and the cytoplasmic tail with either an N-terminal FLAG tag (as also described in Examples 1 and 2) or with a C-terminal myc-tag (in Example 8 referred to as FLAG-mini and mini-myc, respectively; FIG. 11A). Apart from these membrane-bound minireceptors, we also produced soluble forms of the minireceptor containing two, four, and six 3Fn-domains (s3Fn1-2, s3Fn1-4, and s3Fn1-6, respectively) but omitting the cytoplasmic tail and the transmembrane region.

First, we tested the ability of the two membrane-bound minireceptors to form a complex using co-immunoprecipitation analysis of cells transfected with FLAG-mini and mini-myc. We could clearly precipitate mini-myc using an anti-FLAG antibody, when both minireceptors were co-expressed, while cells that were only transfected with mini-myc but not expressing FLAG-mini showed no precipitation of mini-myc (FIG. 11B). This demonstrates that the two minireceptors form a complex, but does not exclude whether this could be mediated by the cytoplasmic tails that could be part of larger cytosolic complexes.

In a second set of experiments when testing the expression of the s3Fn1-6 construct, we observed a clear signal corresponding to a dimer in WB analysis after normal SDS-PAGE separation of proteins from both lysates and medium from cells transfected with the soluble, six-domain SORLA fragment (FIG. 1C). This dimer formation was sensitive to treatment of the samples with b-mercaptoethanol (β-ME) reducing intradomain disulfide-bridges present in the first and sixth 3Fn-domain, indicative that the dimer formation depends on the structural integrity of correctly folded 3Fn-domains, and that this is independent on the SORLA™ and tail domains.

Finally, we also tested whether the minireceptor could interact with the full-length form of SORLA, forming heterodimers. We co-transfected HEK293 cells with FLAG-mini and full-length SORLA, prepared cell lysates, and precipitated proteins using anti-FLAG antibody. WB analysis using detection by a polyclonal serum against the SORLA extracellular fragment clearly showed that FLAG-mini also precipitates full-length SORLA in cells (FIG. 1D). Lysates from HEK293 cells co-transfected with FLAG-mini and an empty vector or double transfected cells treated in parallel but leaving out the anti-FLAG antibody in the IP served as negative control experiments.

### Cellular Analysis of SORLA Dimerization in Endosomes

We next turned our attention to study the cellular localization of SORLA dimer formation. We prepared constructs for full-length SORLA proteins with C-terminal FLAG- or GFP-tags (FIG. 12A), and applied the proximity ligation assay (PLA) to study where in cells SORLA form dimers.

An immunostaining was performed using secondary antibodies tagged with short oligonucleotides able to hybridize when the two target molecules get into close proximity (i.e. dimerizes) allowing for rolling-circle amplification. For visualization by confocal microscopy fluorescently labeled oligos are bound to complementary sequences on the amplicon represented by red dots. Strong PLA signals were detected by microscopy when staining with both a FLAG and a GFP antibody simultaneously indicating dimerization between the two SORLA molecules.

First, we tested whether the two different tags affected the localization of the tagged receptor forms using double transfected HEK293 cells and determination of their degree of co-localization. From this experiment we observed no indication that the tags lead to differences in cellular localization as they showed a strong overlap and Mander's coefficient of 0.71 (FIG. 12B). Next, we performed control experiments for the PLA setup but leaving out either the anti-FLAG (FIG. 12C) or anti-GFP (FIG. 12D) that in both settings abolished formation of the PLA signal. However, in the presence of both antibodies, we observed a strong PLA signal (FIG. 12E) confirming that two full-length SORLA proteins can form a dimer within cells. However, when we determined the degree of co-localization between the PLA signal (representing SORLA dimers) and SORLA-GFP (representing both monomers and dimers), we noticed a clear lack of co-localization for several PLA signals and Mander's coefficient confirmed an overlap of only 0.35 (FIG. 12E), suggesting that only a fraction of the cellular pool of SORLA protein exist as dimers.

We and others have previously demonstrated how SORLA associates with retromer in endosomes (Fjorback et al. 2012), and is has been shown how the related receptor Sortilin forms a dimer specifically in acidic compartments (Jauliene et al. 2017). We therefore hypothesized that also SORLA dimers are predominantly formed in the endosome. Co-staining for the PLA signal representing dimers and the VPS35 subunit of the retromer core showed a high degree of co-localization with Mander's coefficient of 0.70, confirming that the majority of SORLA dimers are located together with retromer (FIG. 12F).

### Endosomal Retromer Binding to a SORLA Dimer

We next wanted to confirm that cellular SORLA exists in equilibrium between monomer and dimer using the bimolecular fluorescence complementation (BiFC) assay as an independent approach.

This technique is based upon structural complementation between non-fluorescent N-terminal and C-terminal fragments of a split fluorescent protein (such as GFP) fused to bait and prey proteins, respectively. Upon interaction between the bait and prey proteins the split domains get into close proximity enabling them to refold into a functional β-barrel structure containing the fluorophore (FIG. 13A). Thereby, dimerization of the two proteins, in our case SORLA, drive the formation of a fluorescent protein that can be visualized by fluorescent microscopy.

Accordingly, two SORLA constructs were engineered to contain C-terminal fusions of either the N-terminal (Met¹-Gln¹⁵⁷) or C-terminal (Lys¹⁵⁸-Lys²³⁸) fragment of the venus fluorescent protein (a derivative of GFP). These two SORLA constructs were termed SORLA-V1 and SORLA-V2 containing either the N-terminal or C-terminal part of the venus fluorescent protein, respectively.

Transfection of HEK293T cells with each of the two SORLA BiFC constructs individually did not produce any green fluorescent signals (FIG. 13C, upper and middle row, Venus). In contrast, co-transfection with both constructs revealed specific green cytoplasmic signals indicating that SORLA dimerization has occurred (FIG. 13C, lower row,*). Simultaneous staining with a SORLA antibody showed a predominant perinuclear localization of the SORLA molecules (arrow) in agreement with previous studies demonstrating that SORLA is mainly localized to the perinuclear region of HEK293 cells (Herskowitz et al. 2012). Quantification of the overlap between the green venus signal specific for SORLA dimers and the total SORLA pool identified by the anti-SORLA staining confirmed that only a fraction of SORLA exist as dimers (Mander's coefficient 0.37) (FIG. 13D).

We next wanted to test if retromer binds preferentially to the SORLA monomer or dimer. For that purpose, we applied the GFP-trap technique that rely on a nanobody that interacts with the venus protein (as well as GFP) in their folded state. However, the nanobody used for the GFP-trap does not bind neither the V1 nor the V2 with sufficient affinity to allow precipitation of these isolated parts of the venus protein.

We transfected cells with SORLA-GFP, SORLA-V1, SORLA-V2, combined SORLA-V1 and SORLA-V2, and full-length SORLA without tags, and confirmed that SORLA forms a dimer that can be precipitated with the GFP-trap (FIG. 13B). As anticipated also SORLA-GFP is precipitated using the GFP-trap, representing both monomer and dimer SORLA, whereas the precipitate from the reconstituted venus protein will only contain the SORLA dimer fraction. By reprobe of the membrane with an antibody against the VPS26b subunit of retromer, we could demonstrate that the endosomal SORLA dimer fraction indeed exists in complex with retromer (FIG. 13B). This tool will allow for future analysis of the interactome of SORLA monomer and dimers, but has to this end confirmed that SORLA forms a dimer in endosomes where it forms a complex with retromer.

### Conclusion

Combined, our data provides the first evidence of SORLA dimer formation by its 3Fn-domains. Further, the data further supports that the minireceptor is sufficient, and even superior in certain regards compared to full-length SorLA (e.g. in regard to its effect on non-amyloidogenic and amyloidogenic processing) to interact with retromer.

Sequences

SEQ ID NO: 1
DNA sequence of full mini-receptor construct
BamHI + 3X-FLAG-TAG + 6xFN3 + transmembrane domain + cytoplasmic tail + stop
codon + EcoRI
CAGGATCCGACTACAAGGACCACGACGGCGACTACAAGGACCACGACATCGACTACAAGG
ACGACGACGACAAGGAGTTGACTGTGTACAAAGTACAGAATCTTCAGTGGACAGCTGACTT
CTCTGGGGATGTGACTTTGACCTGGATGAGGCCCAAAAAAATGCCCTCTGCTTCTTGTGTA
TATAATGTCTACTACAGGGTGGTTGGAGAGAGCATATGGAAGACTCTGGAGACCCACAGC
AATAAGACAAACACTGTATTAAAAGTCTTGAAACCAGATACCACGTATCAGGTTAAAGTACA
GGTTCAGTGTCTCAGCAAGGCACACAACACCAATGACTTTGTGACCCTGAGGACCCCAGA
GGGATTGCCAGATGCCCCTCGAAATCTCCAGCTGTCACTCCCCAGGGAAGCAGAAGGTGT
GATTGTAGGCCACTGGGCTCCTCCCATCCACACCCATGGCCTCATCCGTGAGTACATTGTA
GAATACAGCAGGAGTGGTTCCAAGATGTGGGCCTCCCAGAGGGCTGCTAGTAACTTTACA
GAAATCAAGAACTTATTGGTCAACACTCTATACACCGTCAGAGTGGCTGCGGTGACTAGTC
GTGGAATAGGAAACTGGAGCGATTCTAAATCCATTACCACCATAAAAGGAAAAGTGATCCC
ACCACCAGATATCCACATTGACAGCTATGGTGAAAATTATCTAAGCTTCACCCTGACCATG
GAGAGTGATATCAAGGTGAATGGCTATGTGGTGAACCTTTTCTGGGCATTTGACACCCACA
AGCAAGAGAGGAGAACTTTGAACTTCCGAGGAAGCATATTGTCACACAAAGTTGGCAATCT
GACAGCTCATACATCCTATGAGATTTCTGCCTGGGCCAAGACTGACTTGGGGGATAGCCCT
CTGGCATTTGAGCATGTTATGACCAGAGGGGTTCGCCCACCTGCACCTAGCCTCAAGGCC
AAAGCCATCAACCAGACTGCAGTGGAATGTACCTGGACCGGCCCCCGGAATGTGGTTTAT
GGTATTTTCTATGCCACGTCCTTTCTTGACCTCTATCGCAACCCGAAGAGCTTGACTACTTC
ACTCCACAACAAGACGGTCATTGTCAGTAAGGATGAGCAGTATTTGTTTCTGGTCCGTGTA
GTGGTACCCTACCAGGGGCCATCCTCTGACTACGTTGTAGTGAAGATGATCCCGGACAGC
AGGCTTCCACCCCGTCACCTGCATGTGGTTCATACGGGCAAAACCTCCGTGGTCATCAAG
TGGGAATCACCGTATGACTCTCCTGACCAGGACTTGTTGTATGCAATTGCAGTCAAAGATC
TCATAAGAAAGACTGACAGGAGCTACAAAGTAAAATCCCGTAACAGCACTGTGGAATACAC
CCTTAACAAGTTGGAGCCTGGCGGGAAATACCACATCATTGTCCAACTGGGGAACATGAG
CAAAGATTCCAGCATAAAAATTACCACAGTTTCATTATCAGCACCTGATGCCTTAAAAATCA
TAACAGAAAATGATCATGTTCTTCTGTTTTGGAAAAGCCTGGCTTTAAAGGAAAAGCATTTT
AATGAAAGCAGGGGCTATGAGATACACATGTTTGATAGTGCCATGAATATCACAGCTTACC
TTGGGAATACTACTGACAATTTCTTTAAAATTTCCAACCTGAAGATGGGTCATAATTACACG
TTCACCGTCCAAGCAAGATGCCTTTTTGGCAACCAGATCTGTGGGGAGCCTGCCATCCTG
CTGTACGATGAGCTGGGGTCTGGTGCAGATGCATCTGCAACGCAGGCTGCCAGATCTACG
GATGTTGCTGCTGTGGTGGTGCCCATCTTATTCCTGATACTGCTGAGCCTGGGGGTGGGG
TTTGCCATCCTGTACACGAAGCACCGGAGGCTGCAGAGCAGCTTCACCGCCTTCGCCAAC
AGCCACTACAGCTCCAGGCTGGGGTCCGCAATCTTCTCCTCTGGGGATGACCTGGGGGAA
GATGATGAAGATGCCCCTATGATAACTGGATTTTCAGATGACGTCCCCATGGTGATAGCCT
GAGAATTCGT
SEQ ID NO: 2
DNA sequence of the FnIII-1 domain
GAGTTGACTGTGTACAAAGTACAGAATCTTCAGTGGACAGCTGACTTCTCTGGGGATGTGA
CTTTGACCTGGATGAGGCCCAAAAAAATGCCCTCTGCTTCTTGTGTATATAATGTCTACTAC
AGGGTGGTTGGAGAGAGCATATGGAAGACTCTGGAGACCCACAGCAATAAGACAAACACT
GTATTAAAAGTCTTGAAACCAGATACCACGTATCAGGTTAAAGTACAGGTTCAGTGTCTCAG
CAAGGCACACAACACCAATGACTTTGTGACCCTGAGGACCCCAGAGGGA
SEQ ID NO: 3
DNA sequence of the FnIII-2 domain
TTGCCAGATGCCCCTCGAAATCTCCAGCTGTCACTCCCCAGGGAAGCAGAAGGTGTGATT
GTAGGCCACTGGGCTCCTCCCATCCACACCCATGGCCTCATCCGTGAGTACATTGTAGAAT
ACAGCAGGAGTGGTTCCAAGATGTGGGCCTCCCAGAGGGCTGCTAGTAACTTTACAGAAA
TCAAGAACTTATTGGTCAACACTCTATACACCGTCAGAGTGGCTGCGGTGACTAGTCGTGG
AATAGGAAACTGGAGCGATTCTAAATCCATTACCACCATAAAAGGAAAA
SEQ ID NO: 4
DNA sequence of the FnIII-3 domain
GTGATCCCACCACCAGATATCCACATTGACAGCTATGGTGAAAATTATCTAAGCTTCACCCT
GACCATGGAGAGTGATATCAAGGTGAATGGCTATGTGGTGAACCTTTTCTGGGCATTTGAC
ACCCACAAGCAAGAGAGGAGAACTTTGAACTTCCGAGGAAGCATATTGTCACACAAAGTTG
GCAATCTGACAGCTCATACATCCTATGAGATTTCTGCCTGGGCCAAGACTGACTTGGGGGA
TAGCCCTCTGGCATTTGAGCATGTTATGACCAGA
SEQ ID NO: 5
DNA sequence of the FnIII-4 domain
GGGGTTCGCCCACCTGCACCTAGCCTCAAGGCCAAAGCCATCAACCAGACTGCAGTGGAA
TGTACCTGGACCGGCCCCCGGAATGTGGTTTATGGTATTTTCTATGCCACGTCCTTTCTTG
ACCTCTATCGCAACCCGAAGAGCTTGACTACTTCACTCCACAACAAGACGGTCATTGTCAG
TAAGGATGAGCAGTATTTGTTTCTGGTCCGTGTAGTGGTACCCTACCAGGGGCCATCCTCT
GACTACGTTGTAGTGAAGATGATCCCGGACAGC
SEQ ID NO: 6
DNA sequence of the FnIII-5 domain
AGGCTTCCACCCCGTCACCTGCATGTGGTTCATACGGGCAAAACCTCCGTGGTCATCAAG
TGGGAATCACCGTATGACTCTCCTGACCAGGACTTGTTGTATGCAATTGCAGTCAAAGATC
TCATAAGAAAGACTGACAGGAGCTACAAAGTAAAATCCCGTAACAGCACTGTGGAATACAC
CCTTAACAAGTTGGAGCCTGGCGGGAAATACCACATCATTGTCCAACTGGGGAACATGAG
CAAAGATTCCAGCATAAAAATTACCACAGTTTCATTA
SEQ ID NO: 7
DNA sequence of the FnIII-6 domain
TCAGCACCTGATGCCTTAAAAATCATAACAGAAAATGATCATGTTCTTCTGTTTTGGAAAAG
CCTGGCTTTAAAGGAAAAGCATTTTAATGAAAGCAGGGGCTATGAGATACACATGTTTGATA
GTGCCATGAATATCACAGCTTACCTTGGGAATACTACTGACAATTTCTTTAAAATTTCCAAC
CTGAAGATGGGTCATAATTACACGTTCACCGTCCAAGCAAGATGCCTTTTTGGCAACCAGA
TCTGTGGGGAGCCTGCCATCCTGCTGTACGATGAGCTGGGGTCT
SEQ ID NO: 8
DNA sequence of the Transmembrane domain
GGTGCAGATGCATCTGCAACGCAGGCTGCCAGATCTACGGATGTTGCTGCTGTGGTGGTG
CCCATCTTATTCCTGATACTGCTGAGCCTGGGGGTGGGGTTTGCCATCCTGTACACG
SEQ ID NO: 9
DNA sequence of the Cytoplasmic tail
AAGCACCGGAGGCTGCAGAGCAGCTTCACCGCCTTCGCCAACAGCCACTACAGCTCCAG
GCTGGGGTCCGCAATCTTCTCCTCTGGGGATGACCTGGGGGAAGATGATGAAGATGCCCC
TATGATAACTGGATTTTCAGATGACGTCCCCATGGTGATAGCCTGA
SEQ ID NO: 10
Amino acid sequence of full mini-receptor construct (with FLAG-tag)
DYKDHDGDYKDHDIDYKDDDDKELTVYKVQNLQWTADFSGDVTLTWMRPKKMPSASCVYNV
YYRWGESIWKTLETHSNKTNTVLKVLKPDTTYQVKVQVQCLSKAHNTNDFVTLRTPEGLPDA
PRNLQLSLPREAEGVIVGHWAPPIHTHGLIREYIVEYSRSGSKMWASQRAASNFTEIKNLLVNTL
YTVRVAAVTSRGIGNWSDSKSITTIKGKVIPPPDIHIDSYGENYLSFTLTMESDIKVNGYVVNLFW
AFDTHKQERRTLNFRGSILSHKVGNLTAHTSYEISAWAKTDLGDSPLAFEHVMTRGVRPPAPSL
KAKAINQTAVECTWTGPRNVVYGIFYATSFLDLYRNPKSLTTSLHNKTVIVSKDEQYLFLVRVVV
PYQGPSSDYVWVKMIPDSRLPPRHLHVVHTGKTSVVIKWESPYDSPDQDLLYAIAVKDLIRKTD
RSYKVKSRNSTVEYTLNKLEPGGKYHIIVQLGNMSKDSSIKITTVSLSAPDALKIITENDHVLLFW
KSLALKEKHFNESRGYEIHMFDSAMNITAYLGNTTDNFFKISNLKMGHNYTFTVQARCLFGNQI
CGEPAILLYDELGSGADASATQAARSTDVAAVVVPILFLILLSLGVGFAILYTKHRRLQSSFTAFA
NSHYSSRLGSAIFSSGDDLGEDDEDAPMITGFSDDVPMVIA
SEQ ID NO: 11
Amino acid sequence of the FnIII-1 domain
ELTVYKVQNLQWTADFSGDVTLTWMRPKKMPSASCVYNVYYRVVGESIWKTLETHSNKTNTV
LKVLKPDTTYQVKVQVQCLSKAHNTNDFVTLRTPEG
SEQ ID NO: 12
Amino acid sequence of the FnIII-2 domain
LPDAPRNLQLSLPREAEGVIVGHWAPPIHTHGLIREYIVEYSRSGSKMWASQRAASNFTEIKNLL
VNTLYTVRVAAVTSRGIGNWSDSKSITTIKGK
SEQ ID NO: 13
Amino acid sequence of the FnIII-3 domain
VIPPPDIHIDSYGENYLSFTLTMESDIKVNGYVVNLFWAFDTHKQERRTLNFRGSILSHKVGNLT
AHTSYEISAWAKTDLGDSPLAFEHVMTR
SEQ ID NO: 14
Amino acid sequence of the FnIII-4 domain
GVRPPAPSLKAKAINQTAVECTWTGPRNVVYGIFYATSFLDLYRNPKSLTTSLHNKTVIVSKDE
QYLFLVRVVVPYQGPSSDYVVVKMIPDS
SEQ ID NO: 15
Amino acid sequence of the FnIII-5 domain
RLPPRHLHVVHTGKTSVVIKWESPYDSPDQDLLYAIAVKDLIRKTDRSYKVKSRNSTVEYTLNKL
EPGGKYHIIVQLGNMSKDSSIKITTVSL
SEQ ID NO: 16
Amino acid sequence of the FnIII-6 domain
SAPDALKIITENDHVLLFWKSLALKEKHFNESRGYEIHMFDSAMNITAYLGNTTDNFFKISNLKM
GHNYTFTVQARCLFGNQICGEPAILLYDELGS
SEQ ID NO: 17
Amino acid sequence of the Transmembrane domain
GADASATQAARSTDVAAVVVPILFLILLSLGVGFAILYT
SEQ ID NO: 18
Amino acid sequence of the Cytoplasmic tail
KHRRLQSSFTAFANSHYSSRLGSAIFSSGDDLGEDDEDAPMITGFSDDVPMVIA
SEQ ID NO: 19
Forward primer
CAGGATCCGACTACAAGGACCACGACGGCGACTACAAGGACCACGACATCGACT
ACAAGGACGACGACGACAAGGAGTTGACTGTGTACAAAGTACAG
SEQ ID NO: 20
Reverse primer
GTGAATTCTCAGGCTATCACCATGG
SEQ ID NO: 21
Flag tag
DYKDHDGDYKDHDIDYKDDDDK
SEQ ID NO: 22
Amino acid sequence of full mini-receptor construct (without the Flag-tag)
ELTVYKVQNLQWTADFSGDVTLTWMRPKKMPSASCVYNVYYRVVGESIWKTLETHSNKTNTV
LKVLKPDTTYQVKVQVQCLSKAHNTNDFVTLRTPEGLPDAPRNLQLSLPREAEGVIVGHWAPPI
HTHGLIREYIVEYSRSGSKMWASQRAASNFTEIKNLLVNTLYTVRVAAVTSRGIGNWSDSKSITT
IKGKVIPPPDIHIDSYGENYLSFTLTMESDIKVNGYVVNLFWAFDTHKQERRTLNFRGSILSHKV
GNLTAHTSYEISAWAKTDLGDSPLAFEHVMTRGVRPPAPSLKAKAINQTAVECTWTGPRNVVY
GIFYATSFLDLYRNPKSLTTSLHNKTVIVSKDEQYLFLVRVVVPYQGPSSDYVVVKMIPDSRLPP
RHLHVVHTGKTSVVIKWESPYDSPDQDLLYAIAVKDLIRKTDRSYKVKSRNSTVEYTLNKLEPG
GKYHIIVQLGNMSKDSSIKITTVSLSAPDALKIITENDHVLLFWKSLALKEKHFNESRGYEIHMFD
SAMNITAYLGNTTDNFFKISNLKMGHNYTFTVQARCLFGNQICGEPAILLYDELGSGADASATQ
AARSTDVAAVVVPILFLILLSLGVGFAILYTKHRRLQSSFTAFANSHYSSRLGSAIFSSGDDLGED
DEDAPMITGFSDDVPMVIA

### REFERENCES

Andersen O M, Reiche R, Schmidt V, Gotthardt M, Spoelgen R, Behlke J, et al. SorLA/LR11, a neuronal sorting receptor that regulates processing of the amyloid precursor protein. Proc Natl Acad Sci USA. 2005; 102(38):13461-6.
Fjorback A W, Seaman M, Gustafsen C, Mehmedbasic A, Gokool S, Wu C, et al.
Retromer binds the FANSHY sorting motif in sorLA to regulate amyloid precursor protein sorting and processing. J Neurosci. 2012; 32(4): 1467-80.
Haass C, Kaether C, Thinakaran G, Sisodia S. Trafficking and proteolytic processing of APP. Cold Spring Harb Perspect Med. 2012; 2(5):a006270.
Herskowitz J H, Offe K, Deshpande A, Kahn R A, Levey A I, Lah J J, GGA1-mediated endocytic traffic of LR11/SorLA alters APP intracellular distribution and amyloid-β production, Mol Biol Cell. 2012 July; 23(14):2645-57. Epub 2012 May 23.
Januliene D, Andersen J L, Nielsen J A, Quistgaard E M, Hansen M, Strandbygaard D, Moeller A, Petersen C M, Madsen P, Thirup S S, Acidic Environment Induces Dimerization and Ligand Binding Site Collapse in the Vps10p Domain of Sortilin, 2017 Dec. 5; 25(12): 1809-1819.e3.
Kitago Y, Nagae M, Nakata Z, Yagi-Utsumi M, Takagi-Niidome S, Mihara E, et al. Structural basis for amyloidogenic peptide recognition by sorLA. Nat Struct Mol Biol. 2015; 22(3): 199-206.
Knupp A, Mishra S, Martinez R, Braggin J E, Szabo M, Kinoshita C, et al. Depletion of the A D Risk Gene SORL1 Selectively Impairs Neuronal Endosomal Traffic Independent of Amyloidogenic APP Processing. Cell Rep. 2020; 31(9): 107719.
Kwart D, Gregg A, Scheckel C, Murphy E A, Paquet D, Duffield M, et al. A Large Panel of Isogenic APP and PSEN1 Mutant Human iPSC Neurons Reveals Shared Endosomal Abnormalities Mediated by APP beta-CTFs, Not Abeta. Neuron. 2019; 104(2):256-70 e5.
Mecozzi V J, Berman D E, Simoes S, Vetanovetz C, Awal M R, Patel V M, et al. Pharmacological chaperones stabilize retromer to limit APP processing. Nat Chem Biol. 2014; 10(6):443-9.
Mehmedbasic A, Christensen S K, Nilsson J, Rüetschi U, Gustafsen C, Poulsen A S, et al. SorLA Complement-type Repeat Domains Protect the Amyloid Precursor Protein against Processing. J Biol Chem. 2015; 290(6):3359-76.
Scheltens P, De Strooper B, Kivipelto M, Holstege H, Chetelat G, Teunissen C E, et al. Alzheimer's disease. Lancet. 2021.
Spoelgen R, von Arnim C A, Thomas A V, Peltan I D, Koker M, Deng A, et al. Interaction of the cytosolic domains of sorLA/LR11 with the amyloid precursor protein (APP) and b-secretase b-site APP-cleaving enzyme. J Neurosci. 2006; 26(2):418-28.

1. A polynucleotide construct encoding upon expression a polypeptide P, wherein the polynucleotide comprises or consists of
a. a first polynucleotide encoding upon expression a first polypeptide Q1 comprising or consisting of one or more amino acid sequences selected from the group consisting of SEQ ID NO: 11, SEQ ID NO: 12, SEQ ID NO: 13, SEQ ID NO: 14, SEQ ID NO: 15 and SEQ ID NO: 16, or a biologically active sequence variant thereof wherein the variant is at least 80%, such as at least 90%, such as at least 95%, such as at least 99% identical to said amino acid sequence of SEQ ID NO: 11, SEQ ID NO: 12, SEQ ID NO: 13, SEQ ID NO: 14, SEQ ID NO: 15 or SEQ ID NO: 16; and
b. a second polynucleotide encoding upon expression a second polypeptide Q2 comprising or consisting of the amino acid sequence of SEQ ID NO: 17, or a sequence which is at least 80% identical to SEQ ID: 17, such as at least 90%, such as at least 95%, such as at least 99%; and
c. a third polynucleotide encoding upon expression a third polypeptide Q3 comprising or consisting of the amino acid sequence of SEQ ID NO: 18, or a sequence which is at least 70% identical to SEQ ID: 18, such as at least 75%, such as at least 80%, such as at least 90%, such as at least 95%, such as at least 99%;
wherein the polypeptide P comprises no more than 700 amino acids.

2. The construct according to claim 1, wherein P comprises or consists of
a. a FnIII cassette of SorLA comprising or consisting of amino acid sequences of SEQ ID NO: 11, SEQ ID NO: 12, SEQ ID NO: 13, SEQ ID NO: 14, SEQ ID NO: 15 and SEQ ID NO: 16, or
an FnIII domain selected from FnIII-1, FnIII-2, FnIII-3, FnIII-4, FnIII-5 or FnIII-6, corresponding to the amino acid sequences of SEQ ID NO: 11, SEQ ID NO: 12, SEQ ID NO: 13, SEQ ID NO: 14, SEQ ID NO: 15 or SEQ ID NO: 16, or any combination thereof; and
b. a transmembrane domain of SorLA comprising or consisting of amino acid sequences of SEQ ID NO: 17, and
c. a cytoplasmic tail domain of SorLA comprising or consisting of amino acid sequences of SEQ ID NO: 18.

3. The construct according to any one of the preceding claims, wherein P is no longer than 685 amino acids.

4. The construct according any one of the preceding claims, wherein P further comprises the amino acid sequence of SEQ ID NO: 21.

5. The construct according to any one of the preceding claims, wherein P comprises or consists of the amino acid sequences of SEQ ID NO: 11, SEQ ID NO: 12, SEQ ID NO: 13, SEQ ID NO: 14, SEQ ID NO: 15, SEQ ID NO: 16, SEQ ID NO: 17 and SEQ ID NO: 18.

6. The construct according to any one of the preceding claims, wherein P comprises or consists of the amino acid sequence of SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18 and SEQ ID NO: 21.

7. A polypeptide P, comprising or consisting of
a. a first polypeptide Q1 comprising or consisting of one or more amino acid sequences selected from the group consisting of SEQ ID NO: 11, SEQ ID NO: 12, SEQ ID NO: 13, SEQ ID NO: 14, SEQ ID NO: 15 and SEQ ID NO: 16, or a biologically active sequence variant thereof wherein the variant is at least 80%, such as at least 90%, such as at least 95%, such as at least 99% identical to said amino acid sequence of SEQ ID NO: 11, SEQ ID NO: 12, SEQ ID NO: 13, SEQ ID NO: 14, SEQ ID NO: 15 or SEQ ID NO: 16; and
b. a second polypeptide Q2 comprising or consisting of the amino acid sequence of SEQ ID NO: 17, or a sequence which is at least 80% identical to SEQ ID: 17, such as at least 90%, such as at least 95%, such as at least 99%; and
c. a third polypeptide Q3 comprising or consisting of the amino acid sequence of SEQ ID NO: 18, or a sequence which is at least 70% identical to SEQ ID: 18, such as at least 75%, such as at least 80%, such as at least 90%, such as at least 95%, such as at least 99%;
wherein the polypeptide P comprises no more than 700 amino acids.

8. The polypeptide P according to claim 7, wherein the polypeptide P is a fusion protein of polypeptide Q1, polypeptide Q2 and polypeptide Q3.

9. The polypeptide P according to any one of claims 7 to 8, wherein P comprises or consists of
a. a FnIII cassette of SorLA comprising or consisting of amino acid sequences of SEQ ID NO: 11, SEQ ID NO: 12, SEQ ID NO: 13, SEQ ID NO: 14, SEQ ID NO: 15 and SEQ ID NO: 16, or
an FnIII domain selected from FnIII-1, FnIII-2, FnIII-3, FnIII-4, FnIII-5 or FnIII-6, corresponding to the amino acid sequences of SEQ ID NO: 11, SEQ ID NO: 12, SEQ ID NO: 13, SEQ ID NO: 14, SEQ ID NO: 15 or SEQ ID NO: 16, or any combination thereof; and
b. a transmembrane domain of SorLA comprising or consisting of amino acid sequences of SEQ ID NO: 17, and
c. a cytoplasmic tail domain of SorLA comprising or consisting of amino acid sequences of SEQ ID NO: 18.

10. The polypeptide P according to any one of claims 7 to 9, wherein P is no longer than 685 amino acids.

11. The polypeptide P according to any one of claims 7 to 10, wherein P further comprises the amino acid sequence of SEQ ID NO: 21.

12. The polypeptide P according to any one of claims 7 to 11, wherein P comprises or consists of the amino acid sequences of SEQ ID NO: 11, SEQ ID NO: 12, SEQ ID NO: 13, SEQ ID NO: 14, SEQ ID NO: 15, SEQ ID NO: 16, SEQ ID NO: 17 and SEQ ID NO: 18.

13. The polypeptide P according to any one of claims 7 to 12, wherein P comprises or consists of the amino acid sequence of SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18 and SEQ ID NO: 21.

14. A vector comprising the polynucleotide construct according to any claims 1 to 6 and/or encoding the polypeptide P of any of claims 7 to 13.

15. The vector according to claim 14, wherein the vector is selected from the group comprising of viral vectors, plasmids, cosmids and artificial chromosomes.

16. The vector according to claim 14, wherein the vector is a plasmid vector.

17. The vector according to claim 14, wherein the vector is a viral vector.

18. The vector according to claim any one of claims 14 to 17, wherein the vector has a packaging capacity from 1 to 40 kb, for example from 1 to 30 kb, such as from 1 to 20 kb, for example from 1 to 15 kb, such as from 1 to 10, for example from 1 to 8 kb, such as from 2 to 7 kb, for example from 3 to 6 kb, such as from 4 to 5 kb.

19. The vector according to any one of the claims 14 to 18, wherein the viral vector is selected from the group consisting of adeno-associated vector (AAV), lentiviral vector, adenoviral vector and retroviral vector.

20. The vector according to any one of the claims 14 to 19, wherein the viral vector is an adeno associated vector (AAV).

21. A cell comprising the construct according to any one of claims 1 to 6, the polypeptide P according to any one of claims 7 to 13 and/or the vector according to any one of claims 14 to 20.

22. The cell according to claim 21, wherein said cell is selected from the group consisting of eukaryotic cells, preferably mammalian cells, more preferably primate cells, more preferably human cells.

23. The cell according to any one of claims 21 to 22, wherein the cell is a mammalian cell.

24. The cell according to any one of claims 21 to 23, wherein the cell is a neural cell.

25. A composition comprising the construct according to any one of claims 1 to 6, the polypeptide P according to any one of claims 7 to 13, the vector according to any one of claims 14 to 20 and/or the cell according to any one of claims 21 to 24.

26. The composition according to claim 26, wherein the composition is a pharmaceutical composition.

27. The composition according to any one of claims 25 to 26, wherein the composition further comprises a pharmaceutically acceptable carrier.

28. The construct according to any one of claims 1 to 6, the polypeptide P according to any one of claims 7 to 13, the vector according to any one of claims 14 to 20, the cell according to any one of claims 21 to 24 and/or the composition according to any one of claims 25 to 27, for use in medicine.

29. The construct according to any one of claims 1 to 6, the polypeptide P according to any one of claims 7 to 13, the vector according to any one of claims 14 to 20, the cell according to any one of claims 21 to 24 and/or the composition according to any one of claims 25 to 27, for use in the treatment, prevention and/or alleviation of Alzheimer's Disease or a disease or disorder associated with Alzheimer's Disease.

30. The construct, polypeptide P, vector, cell and/or composition for use according to claim 29, wherein the Alzheimer's disease is of a type selected from the group consisting of Early-Onset Alzheimer's Disease, Late-Onset Alzheimer's Disease and Familial Alzheimer's Disease.

31. A method of treating Alzheimer's Disease or a disease or disorder associated with Alzheimer's Disease, the method comprising administering to an individual in need thereof the construct according to any one of claims 1 to 6, the polypeptide P according to any one of claims 7 to 13, the vector according to any one of claims 14 to 20, the cell according to any one of claims 21 to 24 and/or the composition according to any one of claims 25 to 27.

32. Use of the construct according to any one of claims 1 to 6, the polypeptide P according to any one of claims 7 to 13, the vector according to any one of claims 14 to 20, the cell according to any one of claims 21 to 24 and/or the composition according to any one of claims 25 to 27, for the manufacture of a medicament for the treatment, prevention and/or alleviation of Alzheimer's Disease, or a disease or disorder associated with Alzheimer's Disease.

33. A method of increasing sAPPα in a cell, the method comprising introducing into a cell the polynucleotide construct according to any one of claims 1 to 6, the polypeptide P of any one of claims 7 to 13, the vector according to any one of claims 14 to 20 and/or the composition of any one of claims 25 to 27, thus increasing sAPPα in said cell.

34. A method of decreasing Aβ38, Aβ40, and/or Aβ42 in a cell, the method comprising introducing into a cell the polynucleotide construct according to any one of claims 1 to 6, the polypeptide P of any one of claims 7 to 13, the vector according to any one of claims 14 to 20 and/or the composition of any one of claims 25 to 27,
thus decreasing Aβ38, Aβ40 and/or Aβ42 in said cell.
